(19) United States
(12) Patent Application Publication
(10) Pub. No.: US 2025/0127821 A1
(43) Pub. Date: Apr. 24, 2025

(54) PHARMACEUTICAL COMPOSITION AND COSMETIC COMPOSITION

(71) Applicants: Koji TANABE, Palo Alto, CA (US); I Peace, Inc., Palo Alto, CA (US)

(72) Inventor: Koji TANABE, Palo Alto, CA (US)

(73) Assignees: Koji TANABE, Palo Alto, CA (US); I Peace, Inc., Palo Alto, CA (US)

(21) Appl. No.: 18/919,995

(22) Filed: Oct. 18, 2024

(30) Foreign Application Priority Data
Oct. 18, 2023 (JP) 2023-179422
Oct. 16, 2024 (JP) 2024-180946

(51) Int. Cl. A61K 35/545; A61K 8/98; A61P 17/00; A61Q 19/08

(52) U.S. Cl. CPC A61K 35/545; A61K 8/981; A61P 17/00; A61Q 19/08

(57) ABSTRACT

Problem to be Solved

To provide pharmaceutical compositions and cosmetic compositions that effectively utilize a stem cell and stem cell culture media.

Solution

A fat-decomposing agent, agent for promoting differentiation induction of a myotube from a myoblast, prostacyclin production promoter, hyaluronic acid production promoter for a chondrocyte, anti-aging agent, agent for treating or preventing a lifestyle-related disease, or agent for treating or preventing an adult disease, each comprising a stem cell, an extract of a stem cell, a medium of a stem cell, a supernatant of a culture medium during stem cell induction, a supernatant of a culture medium of stem cells, or a secretion of a stem cell.

### TECHNICAL FIELD

The present invention relates to pharmaceutical compositions and cosmetic compositions.

### BACKGROUND ART

Embryonic stem (ES) cells are stem cells established from the early embryos of humans and mice. ES cells possess pluripotency, which enables them to differentiate into all cells present in a living organism. Currently, human ES cells are available for use in cell transplantation therapy for various diseases, such as Parkinson's disease, juvenile diabetes, and leukemia. However, there are obstacles associated with the transplantation of ES cells. Specifically, the transplantation of ES cells may induce immune rejection reactions similar to those that occur following unsuccessful organ transplants. Additionally, the use of ES cells, which are established by destroying human embryos, has been met with considerable criticism and opposition from an ethical standpoint.

Against this background, Professor Shinya Yamanaka of Kyoto University succeeded in establishing induced pluripotent stem (iPS) cells by introducing four genes: OCT3/4, KLF4, c-MYC, and SOX2, into somatic cells. This achievement earned Professor Yamanaka the Nobel Prize in Physiology or Medicine in 2012 (see, for example, Patent Document 1). iPS cells are ideal pluripotent cells that do not involve immune rejection or ethical issues. Therefore, iPS cells are expected to be used in cell transplantation therapy. On the other hand, there are reports of reusing the medium used for culturing iPS cells in pharmaceutical compositions (see, for example, Patent Document 2).

### PRIOR ART DOCUMENTS

### Patent Documents

[Patent Document 1] Japanese Patent No. 4183742
[Patent Document 2] Japanese Patent Publication No. 2016-128396

### SUMMARY OF THE INVENTION

### Problems to be Solved by the Invention

One of the objectives of the present invention is to provide pharmaceutical compositions and cosmetic compositions that make effective use of stem cells and stem cell culture media.

### Means for Solving the Problem

According to one aspect of the present invention, there is provided a fat-decomposing agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an anti-obesity agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an agent for the treatment of obesity containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a fat-decomposing promoting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a dieting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a agent for treating or preventing diabetes containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an agent for promoting differentiation induction of a myotube from a myoblast containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a muscle formation promoting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a muscle mass maintenance agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a muscle mass increasing agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a prostacyclin production promoting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an adenylyl cyclase activator containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a cyclic adenosine monophosphate production promoting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a platelet aggregation inhibitor containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a vasodilator containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a treatment or preventive agent for hypertension containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an agent for treating or preventing arteriosclerosis containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an endothelial cell growth inhibitor containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an agent for promoting a chondrocyte hyaluronic acid production containing a stem cell, an extract of a stem cell, a medium of a stem cell, a supernatant of a culture medium during stem cell induction, a supernatant of a culture medium of a stem cell, or a secretion of a stem cell. The agent for promoting the chondrocyte hyaluronic acid production may further include hyaluronic acid.

According to one aspect of the present invention, there is provided an agent for protecting or promoting a proliferation of a chondrocyte containing a stem cell, an extract of a stem cell, a medium of a stem cell, a supernatant of a culture medium during stem cell induction, a supernatant of a culture medium of a stem cell, or a secretion of a stem cell. The agent for protecting or promoting the proliferation of the chondrocyte may further include hyaluronic acid.

According to one aspect of the present invention, there is provided an agent for treating or preventing a cartilage tissue wound containing a stem cell, an extract of a stem cell, a medium of a stem cell, a supernatant of a culture medium during stem cell induction, a supernatant of a culture medium of a stem cell, or a secretion of a stem cell. The agent for treating or preventing the cartilage tissue wound may further include hyaluronic acid.

According to one aspect of the present invention, there is provided an agent for treating or preventing joint pain containing a stem cell, an extract of a stem cell, a medium of a stem cell, a supernatant of a culture medium during stem cell induction, a supernatant of a culture medium of a stem cell, or a secretion of a stem cell. The agent for treating or preventing the joint pain may further contain hyaluronic acid.

According to one aspect of the present invention, there is provided an anti-aging agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a rejuvenating agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an amplifier for an expression of a rejuvenation factor or a rejuvenation-related factor containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an amplifier for an expression of at least one of GDF11, MANF, TIMP2, SPARCL1, THBS4, NAMPT, and NANOG, containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an expression suppressor for an aging marker containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an expression suppressor for at least one of p53 and p16 containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an agent for treating or preventing a lifestyle-related disease containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an agent for treating or preventing an adult disease containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided a chondrocyte viability enhancer containing a stem cell, an extract of a stem cell, a medium of a stem cell, a supernatant of a culture medium during stem cell induction, a supernatant of a culture medium of a stem cell, or a secretion of a stem cell. The chondrocyte viability enhancer may further contain hyaluronic acid.

According to one aspect of the present invention, there is provided an analgesic containing a stem cell, an extract of a stem cell, a medium of a stem cell, a supernatant of a culture medium during stem cell induction, a supernatant of a culture medium of a stem cell, or a secretion of a stem cell. The analgesic may further contain hyaluronic acid.

According to one aspect of the present invention, there is provided a cartilage regeneration agent containing a stem cell, an extract of a stem cell, a medium of a stem cell, a supernatant of a culture medium during stem cell induction, a supernatant of a culture medium of a stem cell, or a secretion of a stem cell. The cartilage regeneration agent may further contain hyaluronic acid.

According to one aspect of the present invention, there is provided an agent for treating, improving, or preventing a joint, a joint disease, or arthritis containing a stem cell, an extract of a stem cell, a medium of a stem cell, a supernatant of a culture medium during stem cell induction, a supernatant of a culture medium of a stem cell, or a secretion of a stem cell. The agent may further contain hyaluronic acid.

According to one aspect of the present invention, there is provided an inhibitor or alleviator of inflammation containing a stem cell, an extract of a stem cell, a medium of a stem cell, a supernatant of a culture medium during stem cell induction, a supernatant of a culture medium of a stem cell, or a secretion of a stem cell. The inhibitor or alleviator of inflammation may further contain hyaluronic acid.

According to one aspect of the present invention, there is provided an extracellular matrix production promoter containing a stem cell, an extract of a stem cell, a medium of a stem cell, a supernatant of a culture medium during stem cell induction, a supernatant of a culture medium of a stem cell, or a secretion of a stem cell. The extracellular matrix production promoter may further contain hyaluronic acid.

According to one aspect of the present invention, there is provided a telomere-extending agent or telomere-shortening suppressor containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, there is provided an initializer, dedifferentiator, or reprogramming agent for a cell containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a fat-decomposing agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an anti-obesity agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for the treatment of obesity.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a fat-decomposing promoting agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a dieting agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for treating or preventing diabetes.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for promoting differentiation induction of myotube from myoblast.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a muscle formation promoting agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a muscle mass maintenance agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a muscle mass increasing agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a prostacyclin production promoting agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing of an adenylyl cyclase activator.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a cyclic adenosine monophosphate production promoter.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a platelet aggregation inhibitor.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a vasodilator.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for treating or preventing hypertension.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for treating or preventing arteriosclerosis.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an endothelial cell growth inhibitor.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a hyaluronic acid production promoter for a chondrocyte.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a chondrocyte protective agent or growth promoter.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for treating or preventing a cartilage tissue injury.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for treating or preventing joint pain.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an anti-aging agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a rejuvenating agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an amplifier for an expression of a rejuvenation factor or a rejuvenation-related factor.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an amplifier for an expression of at least one of GDF11, MANF, TIMP2, SPARCL1, THBS4, NAMPT, and NANOG.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an expression suppressor for an aging marker.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an expression suppressor for at least one of p53 and p16.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for treating or preventing a lifestyle-related disease.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for treating or preventing an adult disease.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a chondrocyte viability enhancer.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an analgesic.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a cartilage regeneration agent.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an agent for treating, improving, or preventing a joint, a joint disease, or arthritis.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an inflammation inhibitor or alleviator.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an extracellular matrix production promoter.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing a telomere-extending agent or telomere-shortening suppressor.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided in manufacturing an initializer, dedifferentiator, or reprogramming agent for a cell.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a fat decomposing therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for an anti-obesity therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a therapy for improving obesity.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a fat decomposing promotion therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a diet therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a treatment or prevention of diabetes.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a therapy for promoting differentiation induction of a myotube from a myoblast.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a muscle formation promotion therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a muscle mass maintenance therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a muscle mass increasing therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a prostacyclin production promotion therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for an adenylyl cyclase activity therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a cyclic adenosine monophosphate production promotion therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a platelet aggregation inhibition therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a vasodilation therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a therapeutic or preventive treatment for hypertension.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a treatment or prevention of arteriosclerosis.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for an endothelial cell growth inhibition therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a hyaluronic acid production promotion therapy for chondrocyte.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a chondrocyte protection or growth promotion therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a treatment or prevention of a cartilage tissue wound.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a treatment or prevention of joint pain.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for an anti-aging therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a rejuvenation therapy.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for an expression enhancement of a rejuvenation factor or a rejuvenation-related factor.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for an expression enhancement of at least one of GDF11, MANF, TIMP2, SPARCL1, THBS4, NAMPT, and NANOG.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a suppression of aging marker expression.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a suppression of expression of at least one of p53 and p16.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a treatment or prevention of lifestyle-related disease.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a treatment or prevention of adult disease.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for improving a viability of chondrocyte.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for analgesia.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for cartilage regeneration.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a treatment, improvement, or prevention of a joint, a joint disease, or arthritis.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for an inhibition or alleviation of inflammation.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for promoting a production of extracellular matrix.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a telomere elongation or telomere shortening inhibition.

According to one aspect of the present invention, use of a stem cell, an extract of a stem cell, a medium of a stem cell, a supernatant of a culture medium during stem cell induction, a supernatant of a culture medium of a stem cell, or a secretion of a stem cell is provided for a cell reinitialization, dedifferentiation, or reprogramming.

According to one aspect of the present invention, a method of fat decomposing therapy is provided, comprising administering to a subject a fat decomposing agent containing a stem cell, an extract of a stem cell, a medium of a stem cell, a supernatant of a culture medium during stem cell induction, a supernatant of a culture medium of a stem cell, or a secretion of a stem cell. The subject is a human or a non-human animal.

According to one aspect of the present invention, a method of obesity treatment is provided, comprising administering to a subject an anti-obesity agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of improvement therapy for obesity is provided, comprising administering to a subject an agent for the treatment of obesity containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of promoting fat decomposing therapy is provided, comprising administering to a subject a fat-decomposing promoting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of diet therapy is provided, comprising administering to a subject a dieting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for treating or preventing diabetes is provided, comprising administering to a subject an agent for treating or preventing diabetes containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for promoting a differentiation induction of myotube from myoblast is provided, comprising administering to a subject an agent for promoting differentiation induction of a myotube from a myoblast containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of muscle formation promotion therapy is provided, comprising administering to a subject a muscle formation promoting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of muscle mass maintenance therapy is provided, comprising administering to a subject a muscle mass maintenance agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of muscle mass increasing therapy is provided, comprising administering to a subject a muscle mass increasing agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of prostacyclin production promotion therapy is provided, comprising administering to a subject a prostacyclin production promoting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of adenylyl cyclase activation therapy is provided, comprising administering to a subject an adenylyl cyclase activator containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of cyclic adenosine monophosphate production promotion therapy is provided, comprising administering to a subject a cyclic adenosine monophosphate production promoting agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of platelet aggregation inhibition therapy is provided, comprising administering to a subject a platelet aggregation inhibitor containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of vasodilation therapy is provided, comprising administering to a subject a vasodilator containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for treating or preventing hypertension is provided, comprising administering to a subject an agent for treating or preventing hypertension containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for treating or preventing arteriosclerosis is provided, comprising administering to a subject an agent for treating or preventing arteriosclerosis containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of endothelial cell growth inhibition therapy is provided, comprising administering to a subject an endothelial cell growth inhibitor containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of hyaluronic acid production promotion therapy for chondrocyte is provided, comprising administering to a subject a hyaluronic acid production promoter containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of chondrocyte protection or growth promotion therapy is provided, comprising administering to a subject a chondrocyte protective agent or growth promoter containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for treating or preventing a cartilage tissue wound is provided, comprising administering to a subject an agent for treating or preventing a cartilage tissue wound containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for treating or preventing joint pain is provided, comprising administering to a subject an agent for treating or preventing joint pain containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of anti-aging therapy is provided, comprising administering to a subject an anti-aging agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of rejuvenation therapy is provided, comprising administering to a subject a rejuvenating agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for enhancing expression of a rejuvenation factor or a rejuvenation-related factor is provided, comprising administering to a subject an agent for enhancing the expression of a rejuvenation factor or a rejuvenation-related factor containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for enhancing the expression of at least one of GDF11, MANF, TIMP2, SPARCL1, THBS4, NAMPT, and NANOG is provided, comprising administering to a subject an amplifier for an expression of at least one of these factors containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for suppressing the expression of an aging marker is provided, comprising administering to a subject an aging marker expression suppressor containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for suppressing the expression of at least one of p53 and p16 is provided, comprising administering to a subject an expression suppressor for at least one of p53 and p16 containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for treating a lifestyle-related disease is provided, comprising administering to a subject an agent for treating or preventing a lifestyle-related disease containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for treating or preventing an adult disease is provided, comprising administering to a subject an agent for treating or preventing an adult disease containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for improving chondrocyte viability is provided, comprising administering to a subject a chondrocyte viability enhancer containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of analgesia is provided, comprising administering to a subject an analgesic containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of cartilage regeneration is provided, comprising administering to a subject a cartilage regeneration agent containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for treating, improving, or preventing a joint, a joint disease, or arthritis is provided, comprising administering to a subject an agent for treating, improving or preventing a joint, a joint disease, or arthritis containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for the inhibition or alleviation of inflammation is provided, comprising administering to a subject an inflammation inhibitor or alleviator containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for promoting the production of extracellular matrix is provided, comprising administering to a subject an extracellular matrix production promoter containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method for extending telomere or inhibiting telomere shortening is provided, comprising administering to a subject a telomere-extending agent or telomere-shortening suppressor containing a stem cell, an extract of a stem cell, a medium of a stem cell, a supernatant of a culture medium during stem cell induction, a supernatant of a culture medium of a stem cell, or a secretion of a stem cell.

According to one aspect of the present invention, a method of cell reinitialization, dedifferentiation, or reprogramming is provided, comprising administering to a subject a cell reinitialization agent, dedifferentiation agent, or reprogramming agent containing a stem cell, an extract of a stem cell, a medium of a stem cell, a supernatant of a culture medium during stem cell induction, a supernatant of a culture medium of a stem cell, or a secretion of a stem cell.

### Effect of the Invention

According to the present invention, it is possible to provide pharmaceutical compositions and cosmetic compositions that make effective use of stem cells and stem cell culture media.

### BRIEF DESCRIPTION OF THE DRAWINGS

FIG. 1: Photograph of adipocytes according to Example 2.

FIG. 2: Photograph of adipocytes according to Example 2.

FIG. 3: Photograph of adipocytes according to Example 2.

FIG. 4: Table showing the measurement results of free glycerol when 5% test substance was added to adipocyte culture medium in Example 2.

FIG. 5: Graph showing the measurement results of free glycerol when 5% test substance was added to adipocyte culture medium in Example 2.

FIG. 6: Table showing the measurement results of free glycerol when 20% test substance was added to adipocyte culture medium in Example 2.

FIG. 7: Graph showing the measurement results of free glycerol when 20% test substance was added to adipocyte culture medium in Example 2.

FIG. 8: Table showing the measurement results of free glycerol when isoproterenol was added to adipocyte culture medium in Example 2.

FIG. 9: Graph showing the measurement results of free glycerol when isoproterenol was added to adipocyte culture medium in Example 2.

FIG. 10: Photograph of cells stained with anti-MHC antibody according to Example 3.

FIG. 11: Photograph of cells stained with anti-MHC antibody according to Example 3.

FIG. 12: Photograph of cells stained with anti-MHC antibody according to Example 3.

FIG. 13: Table showing the measurement results of differentiation index to myotubes when 5% test substance was added to the culture medium of normal human skeletal myoblasts in Example 3.

FIG. 14: Graph showing the measurement results of differentiation index to myotubes when 5% test substance was added to the culture medium of normal human skeletal myoblasts in Example 3.

FIG. 15: Table showing the measurement results of differentiation index to myotubes when 20% test substance was added to the culture medium of normal human skeletal myoblasts in Example 3.

FIG. 16: Graph showing the measurement results of differentiation index to myotubes when 20% test substance was added to the culture medium of normal human skeletal myoblasts in Example 3.

FIG. 17: Table showing the measurement results of differentiation index to myotubes when insulin was added to the culture medium of normal human skeletal myoblasts in Example 3.

FIG. 18: Graph showing the measurement results of differentiation index to myotubes when insulin was added to the culture medium of normal human skeletal myoblasts in Example 3.

FIG. 19: Table showing the measurement results of 6-keto-prostaglandin Fla when 5% test substance was added to the culture medium of normal human umbilical vein endothelial cells in Example 4.

FIG. 20: Graph showing the amount of 6-keto-prostaglandin Fla when 5% test substance was added to the culture medium of normal human umbilical vein endothelial cells in Example 4.

FIG. 21: Table showing the measurement results of 6-keto-prostaglandin Fla when 20% test substance was added to the culture medium of normal human umbilical vein endothelial cells in Example 4.

FIG. 22: Graph showing the amount of 6-keto-prostaglandin Fla when 20% test substance was added to the culture medium of normal human umbilical vein endothelial cells in Example 4.

FIG. 23: Table showing the measurement results of 6-keto-prostaglandin Fla when arachidonic acid was added to the culture medium of normal human umbilical vein endothelial cells in Example 4.

FIG. 24: Graph showing the amount of 6-keto-prostaglandin Fla when arachidonic acid was added to the culture medium of normal human umbilical vein endothelial cells in Example 4.

FIG. 25: Table showing the measurement results of hyaluronic acid production when 5% test substance was added to the culture medium of human chondrocytes and cultured for 24 hours in Example 5.

FIG. 26: Graph showing the amount of hyaluronic acid production when 5% test substance was added to the culture medium of human chondrocytes and cultured for 24 hours in Example 5.

FIG. 27: Table showing the measurement results of hyaluronic acid production when 20% test substance was added to the culture medium of human chondrocytes and cultured for 24 hours in Example 5.

FIG. 28: Graph showing the amount of hyaluronic acid production when 20% test substance was added to the culture medium of human chondrocytes and cultured for 24 hours in Example 5.

FIG. 29: Table showing the measurement results of hyaluronic acid production when N-acetylglucosamine was added to the culture medium of human chondrocytes and cultured for 24 hours in Example 5.

FIG. 30: Graph showing the amount of hyaluronic acid production when N-acetylglucosamine was added to the culture medium of human chondrocytes and cultured for 24 hours in Example 5.

FIG. 31: Table showing the measurement results of hyaluronic acid production when 5% test substance was added to the culture medium of human chondrocytes and cultured for 72 hours in Example 5.

FIG. 32: Graph showing the amount of hyaluronic acid production when 5% test substance was added to the culture medium of human chondrocytes and cultured for 72 hours in Example 5.

FIG. 33: Table showing the measurement results of hyaluronic acid production when 20% test substance was added to the culture medium of human chondrocytes and cultured for 72 hours in Example 5.

FIG. 34: Graph showing the amount of hyaluronic acid production when 20% test substance was added to the culture medium of human chondrocytes and cultured for 72 hours in Example 5.

FIG. 35: Table showing the measurement results of hyaluronic acid production when N-acetylglucosamine was added to the culture medium of human chondrocytes and cultured for 72 hours in Example 5.

FIG. 36: Graph showing the amount of hyaluronic acid production when N-acetylglucosamine was added to the culture medium of human chondrocytes and cultured for 72 hours in Example 5.

FIG. 37: Table showing the measurement results of hyaluronic acid production when 5% test substance was added to the culture medium of human chondrocytes and cultured for 24 hours in Example 5.

FIG. 38: Graph showing the amount of hyaluronic acid production when 5% test substance was added to the culture medium of human chondrocytes and cultured for 24 hours in Example 5.

FIG. 39: Table showing the measurement results of hyaluronic acid production when 20% test substance was added to the culture medium of human chondrocytes and cultured for 24 hours in Example 5.

FIG. 40 A graph showing the amount of hyaluronic acid produced when 20% of the test substance related to Example 5 was added to a culture medium of human chondrocytes and the culture medium was incubated for 24 hours. A graph is also shown for the amount of hyaluronic acid produced when the culture medium of human chondrocytes was incubated for 72 hours.

FIG. 41 A table showing the measurement results of hyaluronic acid production when 5% of the test substance related to Example 5 was added to the culture medium of human chondrocytes and incubated for 72 hours.

FIG. 42 A graph showing the amount of hyaluronic acid produced when 5% of the test substance related to Example 5 was added to the culture medium of human chondrocytes and incubated for 72 hours.

FIG. 43 A table showing the measurement results of hyaluronic acid production when 20% of the test substance related to Example 5 was added to the culture medium of human chondrocytes and incubated for 72 hours.

FIG. 44 A graph showing the amount of hyaluronic acid produced when 20% of the test substance related to Example 5 was added to the culture medium of human chondrocytes and incubated for 72 hours.

FIG. 45 A table showing the measurement results of human chondrocyte viability when 5% of the test substance related to Example 5 was added to the culture medium of human chondrocytes and incubated for 24 hours.

FIG. 46 A graph showing human chondrocyte viability when 5% of the test substance related to Example 5 was added to the culture medium of human chondrocytes and incubated for 24 hours.

FIG. 47 A table showing the measurement results of human chondrocyte viability when 20% of the test substance related to Example 5 was added to the culture medium of human chondrocytes and incubated for 24 hours.

FIG. 48 A graph showing human chondrocyte viability when 20% of the test substance related to Example 5 was added to the culture medium of human chondrocytes and incubated for 24 hours.

FIG. 49 A table showing the measurement results of human chondrocyte viability when N-acetylglucosamine related to Example 5 was added to the culture medium of human chondrocytes and incubated for 24 hours.

FIG. 50 A graph showing human chondrocyte viability when N-acetylglucosamine related to Example 5 was added to the culture medium of human chondrocytes and incubated for 24 hours.

FIG. 51 A table showing the measurement results of human chondrocyte viability when 5% of the test substance related to Example 5 was added to the culture medium of human chondrocytes and incubated for 72 hours.

FIG. 52 A graph showing human chondrocyte viability when 5% of the test substance related to Example 5 was added to the culture medium of human chondrocytes and incubated for 72 hours.

FIG. 53 A table showing the measurement results of human chondrocyte viability when 20% of the test substance related to Example 5 was added to the culture medium of human chondrocytes and incubated for 72 hours.

FIG. 54 A graph showing human chondrocyte viability when 20% of the test substance related to Example 5 was added to the culture medium of human chondrocytes and incubated for 72 hours.

FIG. 55 A table showing the measurement results of human chondrocyte viability when N-acetylglucosamine related to Example 5 was added to the culture medium of human chondrocytes and incubated for 72 hours.

FIG. 56 A graph showing human chondrocyte viability when N-acetylglucosamine related to Example 5 was added to the culture medium of human chondrocytes and incubated for 72 hours.

FIGS. 57A-57B A microscopic photograph of cells related to the reference example.

FIGS. 58A-58C histogram showing the results of flow cytometry of cells related to the reference example.

FIG. 59 A graph showing the left hind limb weight-bearing ratio of an osteoarthritis model rat related to Example 6.

FIG. 60-64 Graphs showing gene expression levels related to Example 8.

FIG. 65-69 Graphs showing gene expression levels related to Example 8.

FIG. 70-72 Graphs showing gene expression levels related to Example 9.

FIG. 73-77 Graphs showing gene expression levels related to Example 10.

### MODE FOR CARRYING OUT THE INVENTION

The embodiments of the present invention will now be described in detail. The embodiments described hereinafter illustrate apparatuses and methods for realizing the technical concept of the present invention and do not limit the combination of components to the following examples. The technical concept of this invention can be variously modified within the scope of the claims.

A pharmaceutical composition and/or cosmetic composition according to the embodiments includes a stem cell, an extract of a stem cell, a stem cell culture medium, a supernatant of a culture medium during the induction of a stem cell, a supernatant of a stem cell culture medium, or a secretion of a stem cell. The pharmaceutical composition and/or cosmetic composition according to the embodiments may also include at least two or more combinations selected from the extract of the stem cell, the stem cell culture medium, the supernatant of the culture medium during the induction of the stem cell, and the supernatant of the stem cell culture medium.

The pharmaceutical composition and/or cosmetic composition may include, for example, a fat-decomposing agent, an anti-obesity drug, a drug for treating obesity, a fat-decomposing-promoting agent, a dieting agent, an agent for treating or preventing diabetes, an agent for promoting differentiation induction of a myotube from a myoblast, an agent for promoting muscle formation, an agent for maintaining muscle mass, an agent for increasing muscle mass, an agent for promoting a prostacyclin production, an adenylate cyclase activator, an agent for promoting cyclic adenosine monophosphate (cAMP) production, an inhibitor for platelet aggregation, a vasodilator, an agent for treating or preventing hypertension, an agent for treating or preventing arteriosclerosis, an inhibitor of vascular endothelial cell proliferation, an agent for promoting hyaluronic acid production of chondrocyte, an agent for promoting proliferation of chondrocyte, an agent for protecting chondrocyte, an agent for treating or preventing a cartilage tissue wound, an agent for promoting extracellular matrix production, an agent for treating or preventing joint pain, an anti-aging agent, a rejuvenating agent, an expression amplifier for a rejuvenation factor, an expression amplifier for a rejuvenation-related factor, an expression amplifier of at least one of GDF11, MANF, TIMP2, SPARCL1, THBS4, NAMPT, NANOG, and TERT, an aging marker expression suppressor, a cellular senescence marker expression suppressor, a p53 expression suppressor, a p16 expression suppressor, a p53 pathway inhibitor, a p16 pathway inhibitor, a p19-p52 pathway inhibitor, a p16-Rb pathway inhibitor, an agent for extending telomere length, an agent for suppressing telomere-shortening, an agent for initializing a cell, an agent for dedifferentiating a cell, an agent for reprogramming a cell, an agent for treating or preventing a lifestyle disease, an agent for treating or preventing an adult disease, an agent for improving chondrocyte viability, an analgesic, an agent for regenerating a cartilage, and/or an agent for treating, improving, or preventing a joint, a joint disease, or arthritis.

The stem cells may be, for example, pluripotent stem cells. The stem cells may be derived from humans or non-human animals. The pluripotent stem cells include, for example, induced pluripotent stem (iPS) cells and embryonic stem (ES) cells. The “stem cell culture medium” may refer, for instance, to culture media not yet used for stem cell cultivation. The “supernatant of the culture medium during stem cell induction” refers to culture media used during the reprogramming of somatic cells into pluripotent stem cells, such as iPS cells. The “supernatant of stem cell culture medium” refers to culture media used for maintaining the culture of stem cells.

The stem cells, extracts of stem cells, stem cell culture medium, supernatant of culture medium during induction of stem cells, supernatant of stem cell culture medium, or secretions of stem cells may be freeze-dried. Additionally, they may be encapsulated in nano-capsules or contained within nano-emulsions or similar emulsions.

iPS cells may be induced by introducing reprogramming factors, such as OCT3/4, KLF4, c-MYC, and SOX2, into somatic cells, such as differentiated cells of blood cells, fibroblasts, and urinary cells. Induction into iPS cells is also referred to as reprogramming, initialization, transdifferentiation, or lineage reprogramming, as well as cell fate reprogramming.

Pluripotent stem cells may be induced in three-dimensional cultures, such as suspension cultures. During three-dimensional culture, a gel medium or liquid medium may be used, and feeder cells may not be required. The pluripotent stem cells may, for instance, have positive rates of TRA1-60, OCT3/4, SSEA3, SSEA4, TRA1-81, LIN28, and NANOG of at least 30%, 50%, or preferably 80% or more.

The medium used for induction culture can include human ES/iPS media such as TeSR2 (STEMCELL Technologies), though it is not limited to this and various stem cell media can be utilized. Examples include Primate ES Cell Medium, Reprostem, ReproFF, ReproFF2, ReproXF (Reprocell), mTeSR1, TeSRE8, ReproTeSR (STEMCELL Technologies), PluriSTEM™ Human ES/iPS Medium (Merck), NutriStem™ XF/FF Culture Medium for Human iPS and ES Cells, Pluriton Reprogramming Medium (Stemgent), PluriSTEM™, Stemfit AK02N, Stemfit AK03 (Ajinomoto), ESC-Sure™ Serum and Feeder-Free Medium for hESC/iPS (Applied StemCell), L7™ hPSC Culture System (Lonza), Primate ES Cell Medium (ReproCELL), and Puel (I Peace, Inc.). The medium may, for example, be free of activin A.

A gel medium can be prepared, for example, by adding deacetylated gellan gum to the above medium at a final concentration of 0.5 wt % to 0.001 wt %, 0.1 wt % to 0.005 wt %, or 0.05 wt % to 0.01 wt %.

When culturing the stem cells to maintain them in an undifferentiated state, the medium preferably contains at least 10 ng/ml, more preferably at least 40 ng/ml, of b-FGF. Whether stem cells remain in an undifferentiated state can be confirmed, for example, by verifying that the positive rates for TRA1-60, OCT3/4, SSEA3, SSEA4, TRA1-81, LIN28, and NANOG are at least 30%, 50%, or preferably 80% or higher.

The extract of stem cells may be in liquid form. In other words, the extract may be an extraction solution. The extract may also be a paste of stem cells, obtained by grinding the stem cells. The extract may be in the form of a freeze-dried product or a powder, or alternatively, it may be a dissolved form of stem cells.

The culture medium of stem cells, or the supernatant of the stem cell culture medium, contains stem cell secretions. Examples of stem cell secretions include extracellular vesicles, cytokines, chemokines, and growth factors. Extracellular vesicles are vesicles with a lipid bilayer structure and are also referred to as membrane vesicles. Examples of extracellular vesicles include exosomes and microvesicles. The exosomes are granular materials with a diameter of 30 nm to 150 nm, formed by inward budding of the late endosomal membrane, and secreted outside the cell through exocytosis after fusion with the cell membrane. The exosomes contain lipids and proteins from the cell membrane on their surface and nucleic acids and proteins from the interior of the cell inside.

The process of exosome secretion will be explained in more detail. Upon endocytosis within a cell, early endosomes form, followed by late endosomes. The vesicles formed by inward budding of the endosomal membrane are known as intraluminal vesicles (ILVs). Endosomes containing aggregated intraluminal vesicles are called multivesicular bodies (MVBs). When multivesicular bodies fuse with the cell membrane, the intraluminal vesicles in the multivesicular body are secreted outside the cell. The vesicles released outside the cell are exosomes. This secretion process involves ceramides, a critical component in vesicle budding into the endosome membrane; subunits of the ESCRT complex, such as Hrs; and small G-proteins of the Rab family, including Rab11 and Rab35, involved in intracellular transport of vesicles. Additionally, when multivesicular bodies fuse with the cell membrane for exocytosis, SNARE membrane proteins are reportedly involved.

Methods for recovering the exosomes from the culture medium are not specifically limited. For example, the exosomes may be recovered from the culture medium by centrifugation, such as ultracentrifugation or density-gradient centrifugation. In ultracentrifugation, the culture medium may be centrifuged at 10,000-100,000 g for at least 30 minutes to isolate the exosomes. The exosomes may also be recovered by size-exclusion chromatography, co-precipitation with polyethylene glycol (PEG), or immunoprecipitation using antibodies that specifically recognize CD9, CD63, and CD81 to isolate exosomes from serum or culture supernatants.

The exosomes may also be recovered by affinity purification using proteins specifically expressed by the exosomes. For example, phosphatidylserine, a phospholipid, is expressed on the surface of exosomes. Tim4, an exosome receptor expressed in macrophages, binds phosphatidylserine in a calcium-dependent manner. Magnetic beads bound with the extracellular region of Tim4 can be prepared, and the exosomes can be immobilized on the surface of the magnetic beads via the extracellular region of Tim4. The exosomes can then be recovered and isolated from the culture medium by elution with a chelating agent.

Microvesicles, with a diameter of 100 nm to 1000 nm, are generated by outward budding of the cell membrane. Stem cell secretions may include ectosomes, secreted microvesicles, oncosomes, and prostasomes. Examples of stem cell secretions include peptides, proteins, growth factors, proliferative factors, cytokines, and hormones. The proteins may have molecular weights of at least 3 kDa, 10 kDa, or 20 kDa.

The stem cell secretions may be contained in the supernatant of the stem cell culture medium. The pharmaceutical or cosmetic composition according to the embodiments may contain isolated secretions or concentrated secretions. Examples of methods for isolating and concentrating secretions include freeze-drying, ultrafiltration, precipitation and resuspension, and gel filtration. Examples of protein precipitation methods include ammonium sulfate precipitation, trichloroacetic acid precipitation, acetone precipitation, and ammonium acetate-containing methanol precipitation.

Examples of marker proteins for extracellular vesicles include the tetraspanin family (CD9, CD63, CD81), the integrin family (ITGA, ITGB), ESCRT complex-associated proteins (TSG101, ALIX), lipid raft markers (Flotillin-1/2), and heat shock proteins (HSP70). Therefore, extracellular vesicles can also be isolated using antibodies that specifically recognize these marker proteins.

The secretions may be recovered from the supernatant of the stem cell culture medium. They may also be recovered from the supernatant of the culture medium while stem cells are maintained in culture.

The inventors have discovered, for the first time, that the stem cell, the extract of the stem cell, the stem cell culture medium, the supernatant of the culture medium during induction of the stem cell, and the supernatant of the stem cell culture medium can decompose fat. Here, fat decomposition includes both fat burning and fat consumption. Accordingly, the stem cell, the extract of the stem cell, the stem cell culture medium, the supernatant of the culture medium during induction of the stem cell, and the supernatant of the stem cell culture medium can be used as the fat-decomposing agent, the anti-obesity drug, the drug for treating obesity, the agent for promoting fat-decomposing, and the dieting agent. Additionally, in diabetes, an increase in blood fat is known to occur. Therefore, the stem cell, the extract of the stem cell, the stem cell culture medium, the supernatant of the culture medium during induction of the stem cell, and the supernatant of the stem cell culture medium can be used as the agent for treating or preventing diabetes. The fat may include, for example, triglycerides. The diabetes may be type 1 diabetes, type 2 diabetes, diabetes from other specific mechanisms, diseases causing diabetes, or gestational diabetes.

The inventors have also found, for the first time, that the stem cell, the extract of the stem cell, the stem cell culture medium, the supernatant of the culture medium during induction of the stem cell, and the supernatant of the stem cell culture medium promote the differentiation of myoblasts into myotubes. Therefore, these materials can be used as the agent for promoting differentiation induction of a myotube from a myoblast. Additionally, myotubes form muscle fibers and subsequently develop into muscle. Thus, the stem cell, the extract of the stem cell, the stem cell culture medium, the supernatant of the culture medium during induction of the stem cell, and the supernatant of the stem cell culture medium can be used as the agent for promoting muscle formation, the agent for maintaining muscle mass, and the agent for increasing muscle mass.

The inventors have discovered, for the first time, that the stem cell, the extract of the stem cell, the stem cell culture medium, the supernatant of the culture medium during induction of the stem cell, and the supernatant of the stem cell culture medium promote the production of prostacyclin by endothelial cell. Accordingly, the stem cell, the extract of the stem cell, the stem cell culture medium, the supernatant of the culture medium during induction of the stem cell, and the supernatant of the stem cell culture medium can be used as the agent for promoting the prostacyclin production. Prostacyclin, also known as prostaglandin 12 (PGI2), activates adenylate cyclase and promotes the production of cyclic adenosine monophosphate (cAMP). Therefore, these stem cell materials can also be used as the adenylate cyclase activator and the agent for promoting cyclic adenosine monophosphate (cAMP) production.

Additionally, prostacyclin has antiplatelet aggregation activity and dilates vascular smooth muscles. Therefore, the stem cell, the extract of the stem cell, the stem cell culture medium, the supernatant of the culture medium during induction of the stem cell, and the supernatant of the stem cell culture medium can be used as the inhibitor for the platelet aggregation, the vasodilator, the agent for treating or preventing hypertension, and the agent for treating or preventing arteriosclerosis. Furthermore, prostacyclin inhibits the proliferation of vascular endothelial cells. Therefore, these stem cell materials can be used as the inhibitor of vascular endothelial cell proliferation.

The inventors have also discovered, for the first time, that the stem cell, the extract of the stem cell, the stem cell culture medium, the supernatant of the culture medium during induction of the stem cell, and the supernatant of the stem cell culture medium promote hyaluronic acid production by chondrocyte. Accordingly, these materials can be used as the agent for promoting hyaluronic acid production of chondrocyte. Hyaluronic acid protects cartilage and provides lubricating, cushioning, and anti-inflammatory effects for a joint. Therefore, the stem cell, the extract of the stem cell, the stem cell culture medium, the supernatant of the culture medium during induction of the stem cell, and the supernatant of the stem cell culture medium can also be used as the agent for promoting chondrocyte proliferation, the agent for treating or preventing the cartilage tissue injury, the agent for treating or preventing joint pain, the analgesics, the agent for treating, improving, or preventing the joint, the joint disease, or the arthritis, the agent for inhibiting inflammation, and the agent for suppressing inflammation. Examples of the joint diseases include arthrosis, osteoarthritis, temporomandibular joint disorder, femoral head necrosis, rheumatoid arthritis, chondritis, tennis elbow, golfer's elbow, baseball elbow, hernia, cartilage injuries, and meniscus injuries. The arthrosis may include osteoarthritis, for example. The agent for promoting the hyaluronic acid production of chondrocyte, the agent for promoting the chondrocyte proliferation, the agent for treating or preventing the cartilage tissue injury, the agent for treating or preventing the joint pain, the analgesic, and the agent for treating, improving, or preventing the joint, joint disease, or arthritis may additionally contain hyaluronic acid.

Furthermore, the inventors have discovered that the stem cell, the extract of the stem cell, the stem cell culture medium, the supernatant of the culture medium during induction of the stem cell, and the supernatant of the stem cell culture medium activate chondrocyte, enhance chondrocyte survival rate, promote chondrocyte proliferation, and rejuvenate chondrocyte. Thus, these materials can be used as the agent for activating chondrocyte, the agent for enhancing chondrocyte survival rate, the agent for promoting chondrocyte proliferation, the agent for protecting chondrocyte, the agent for rejuvenating chondrocyte, the agent for regenerating cartilage, and the agent for treating, improving, or preventing the joint, joint disease, or arthritis. The agent for activating chondrocyte, the agent for enhancing chondrocyte survival rate, the agent for promoting chondrocyte proliferation, the agent for rejuvenating chondrocyte, the agent for regenerating cartilage, and the agent for treating, improving, or preventing the joint, joint disease, or arthritis may also contain hyaluronic acid.

Anti-obesity action, muscle mass-increasing action, vasodilation action, and cartilage-protective action contribute to the suppression of aging and can lead to the treatment and prevention of lifestyle and adult diseases. Therefore, the stem cell, the extract of the stem cell, the stem cell culture medium, the supernatant of the culture medium during induction of the stem cell, and the supernatant of the stem cell culture medium can be used as the anti-aging agent, the agent for treating or preventing lifestyle-related disease, and/or the agent for treating or preventing adult disease.

Additionally, the inventors have found that the stem cell, the extract of the stem cell, the stem cell culture medium, the supernatant of the culture medium during induction of the stem cell, and the supernatant of the stem cell culture medium amplify the expression of GDF11, MANF, TIMP2, SPARCL1, THBS4, NAMPT, NANOG, and TERT, and inhibit the expression of p53 and p16. GDF11, MANF, TIMP2, SPARCL1, THBS4, NAMPT, NANOG, p53, and p16 are expressed in the cells of the entire body, without particular limitation. Examples of cells include skin cells, such as fibroblasts, cells of the epidermal layer, and cells of the dermal layer. Expression of GDF11, MANF, TIMP2, SPARCL1, THBS4, NAMPT, and NANOG is known to be high in humans and non-human animals when young and to decrease as aging progresses. These factors are also called rejuvenation factors or rejuvenation-related factors. Therefore, by increasing the expression of at least one of GDF11, MANF, TIMP2, SPARCL1, THBS4, NAMPT, and NANOG, it is possible to rejuvenate humans and non-human animals and extend their lifespan. TERT promotes telomere elongation and inhibits telomere shortening. Therefore, the stem cell, the extract of the stem cell, the stem cell culture medium, the supernatant of the culture medium during induction of the stem cell, and the supernatant of the stem cell culture medium can be used as the anti-aging agent, the rejuvenation agent, the expression amplifier for the rejuvenation factor, the expression amplifier for the rejuvenation-related factor, the expression amplifier for GDF11, MANF, TIMP2, SPARCL1, THBS4, NAMPT, and NANOG, the expression amplifier for TERT, the agent for extending telomere, and the agent for suppressing telomere-shortening. Additionally, p53 and p16 are known as markers of cellular aging. Therefore, by increasing the expression of at least one of p53 and p16, it is possible to inhibit cellular aging (senescence). Accordingly, the stem cell, the extract of the stem cell, the stem cell culture medium, the supernatant of the culture medium during induction of the stem cell, and the supernatant of the stem cell culture medium can be used as the anti-aging agent, the rejuvenation agent, the expression suppressor for the aging-related factor, the expression suppressor for the cellular aging-related factor, the expression suppressor for the organismal aging-related factor, the expression suppressor for the aging marker, the expression suppressor for the cellular aging marker, the p53 expression suppressor, the p16 expression suppressor, the p53 pathway inhibitor, the p16 pathway inhibitor, the p19-p52 pathway inhibitor, and the p16-Rb pathway inhibitor.

The pharmaceutical composition and cosmetic composition according to the embodiments contain an effective amount of the stem cells, the extracts of the stem cells, the medium of the stem cells, the supernatant of the culture medium during the stem cell induction, the supernatant of the culture medium of the stem cells, or the secretions of the stem cells. Here, the effective amount refers to an amount sufficient to exhibit efficacy as a pharmaceutical composition or cosmetic composition. The effective amount is appropriately set according to factors such as the age of the patient, the target disease, the presence of other active ingredients, and the amount of other ingredients. The pharmaceutical composition and cosmetic composition according to the embodiments may be repeatedly administered to the affected area or target site. Examples of administration routes for the pharmaceutical composition and cosmetic composition include oral administration, subcutaneous injection, intramuscular injection, intravenous injection, intrathecal injection, sublingual administration, oral mucosal administration, rectal administration, vaginal administration, administration to the eyes, administration to the ears, nasal administration, inhalation, spray administration, and transdermal administration.

The pharmaceutical composition and/or cosmetic composition according to the embodiments may contain pharmaceutically acceptable carriers, excipients, disintegrants, buffers, emulsifiers, suspending agents, analgesics, stabilizers, preservatives, antiseptics, and physiological saline, among others. Examples of excipients include lactose, starch, sorbitol, D-mannitol, and sucrose. Examples of disintegrants include carboxymethylcellulose and calcium carbonate. Examples of buffers include phosphates, citrates, and acetates. Examples of emulsifiers include gum arabic, sodium alginate, and tragacanth. The pharmaceutical composition according to the embodiments may be included in injections and intravenous drip solutions. The pharmaceutical composition according to the embodiments may be injected into the affected area. Furthermore, the pharmaceutical composition according to the embodiments may also be a research reagent.

Examples of suspending agents include glycerin monostearate, aluminum monostearate, methylcellulose, carboxymethylcellulose, hydroxyethylcellulose, and sodium lauryl sulfate. Examples of anesthetics include benzyl alcohol, chlorobutanol, and sorbitol. Examples of stabilizers include propylene glycol and ascorbic acid. Examples of preservatives include phenol, benzalkonium chloride, benzyl alcohol, chlorobutanol, and methylparaben. Examples of antimicrobial agents include benzalkonium chloride, paraoxybenzoic acid, and chlorobutanol.

In addition, the pharmaceutical composition and/or cosmetic composition according to the embodiments may include water, alcohol, surfactants (cationic, anionic, nonionic, and amphoteric surfactants), moisturizers (such as glycerin, 1,3-butylene glycol, propylene glycol, propanediol, pentanediol, polyquaternium, amino acids, urea, pyrrolidone carboxylate, nucleic acids, monosaccharides, and oligosaccharides, as well as their derivatives), thickeners (such as polysaccharides, polyacrylate, carboxyvinyl polymers, polyvinylpyrrolidone, polyvinyl alcohol, chitin, chitosan, alginic acid, carrageenan, xanthan gum, and methylcellulose, along with their derivatives), waxes, petroleum jelly, hydrocarbons, saturated and unsaturated fatty acids, and silicone oils, as well as their derivatives, triglycerides such as tri(caprylic/capric) glyceride and trioctanoin, ester oils like isopropyl stearate, natural oils (such as olive oil, camellia oil, avocado oil, almond oil, cocoa butter, evening primrose oil, grape seed oil, macadamia nut oil, eucalyptus oil, rosehip oil, squalane, orange roughy oil, lanolin, and ceramides), preservatives (such as oxybenzoic acid derivatives, dehydroacetic acid salts, chromophores, sorbic acid, and phenoxyethanol, as well as their derivatives), antimicrobial agents (such as sulfur, triclocarban, salicylic acid, zinc pyrithione, and hinokitiol, along with their derivatives), UV absorbers (such as para-aminobenzoic acid, methoxycinnamate, and their derivatives), anti-inflammatory agents (such as allantoin, bisabolol, ε-aminocaproic acid, acetylphytosphingosine, and glycyrrhizic acid, along with their derivatives), antioxidants (such as tocopherol, BHA, BHT, and astaxanthin, along with their derivatives), chelating agents (such as edetate and hydroxyethane diphosphonic acid, along with their derivatives), plant and animal extracts (such as extracts from Ashitaba, aloe, aged citrus, Scutellaria baicalensis, Phellodendron bark, seaweed, quince, chamomile, licorice, kiwi, cucumber, mulberry, birch, Angelica, garlic, peony, hops, horse chestnut, lavender, rosemary, eucalyptus, milk, various peptides, placenta, royal jelly, Euglena, hydrolyzed Euglena, and Euglena oil, as well as refined or fermented products containing these components), pH adjusters (such as inorganic acids, inorganic acid salts, organic acids, and organic acid salts, along with their derivatives), vitamins (such as vitamins A, B, C, D, ubiquinone, nicotinamide, and their derivatives), fermentation products of yeasts, Aspergillus, and lactic acid bacteria, galactomyces ferment filtrate, skin-whitening agents (such as tranexamic acid, cetyl tranexamic acid hydrochloride, 4-n-butylresorcinol, arbutin, kojic acid, ellagic acid, licorice flavonoid, niacinamide, and vitamin C derivatives), ceramides and ceramide derivatives, anti-wrinkle agents (such as retinol, retinal, and their derivatives, nicotinamide, oligopeptides, and natural or synthetic components with neutrophil elastase inhibition and MMP-1 and MMP-2 inhibition), titanium dioxide, talc, mica, silica, zinc oxide, iron oxides, silicon, and processed powders thereof, all of which may be formulated within the pharmaceutical composition and cosmetic composition according to the embodiments to achieve the intended purpose.

It should be noted that the ingredients that can be added to the pharmaceutical composition and/or cosmetic composition according to the embodiments are not limited to those mentioned above. Any component that can be used in a pharmaceutical or cosmetic composition may be freely selected. When using the pharmaceutical composition and/or cosmetic composition as a poultice, additional base materials (such as kaolin and bentonite) and gelling agents (such as polyacrylate and polyvinyl alcohol) may be included within the range necessary to achieve the desired purpose. When used as a bath additive, suitable amounts of sulfates, bicarbonates, borates, dyes, and moisturizers may be incorporated within the composition to achieve the intended purpose, and prepared as a powder type or liquid type. The pharmaceutical composition and/or cosmetic composition according to the embodiments may also be a skin application composition.

### Example 1

### Example 1: Preparation of Test Substances

Test substances 1 to 7, shown in Table 1, were prepared. Test substance 1 contained only Dulbecco's Modified Eagle Medium (DMEM). Test substance 2 contained only the stem cell medium (Puel, I Peace, Inc.).

TABLE 1

| | |
|---|---|
| Test substance 1 | DMEM only |
| Test substance 2 | Puel only |
| Test substance 3 | Pule used for the induction of human iPS cells |
| Test substance 4 | Puel used for two-dimensional |
| | maintenance culture of human iPS cells |
| Test substance 5 | Puel used for three-dimensional |
| | maintenance culture of human iPS cells |
| Test substance 6 | An extract of human iPS cells |
| Test substance 7 | An extract of porcine iPS cells |

Test substance 3 was prepared as follows. A hematopoietic cell medium was prepared by adding recombinant proteins, specifically FLT3 ligand (PeproTech), recombinant human TPO (PeproTech), recombinant human IL-6 (PeproTech), recombinant human G-CSF (PeproTech), and recombinant human SCF (PeproTech) to a serum-free and animal component-free hematopoietic cell medium (Stemspan ACF, STEMCELL Technologies). Human blood cells (peripheral blood mononuclear cells) at a concentration of 2×10≡cells per well were seeded in each well of a 12-well dish. The 12-well dish was then placed in a CO₂ incubator at 37° C. to allow the cells to undergo suspension culture.

Three days after the start of cell culture, additional hematopoietic cell medium was added to each well as needed. On the sixth day, Oct3/4, SOX2, KLF4, and c-Myc were introduced into the cells in each well, after which hematopoietic cell medium was added to each well, and the cells were cultured on a laminin 511-coated dish used as a stem cell culture substrate.

Starting on the second day post-infection, the culture medium in each well was replaced with stem cell medium, half at a time, every two days. Fourteen days post-infection, it was confirmed that ten or more iPS cell colonies had been induced, and the culture supernatant was collected. The collected supernatant was centrifuged at 400 g to remove cells, and the filtered culture supernatant was designated as test substance 3.

Test substance 4 was prepared as follows. Using stem cell medium (Puel, I Peace, Inc.), human iPS cells were maintained in adherent culture on laminin 511-coated culture dishes. The human iPS cells were passaged weekly. During passaging, the human iPS cells were treated with an ES cell dissociation solution (TrypLE Select, trademarked, Thermo Fisher Scientific).

The maintained human iPS cells were then detached from the adhesive culture dish using TrypLE Select. The detached human iPS cells were seeded onto a dish coated with laminin (Nippi). Using stem cell medium (Puel, I Peace, Inc.) containing 10 μmol/L of ROCK inhibitor, the human iPS cells were cultured for one week with daily medium replacement.

Once confluence reached 40% or higher, the medium was refreshed, and two days later, the supernatant of the stem cell medium was collected. The collected supernatant was centrifuged at 400 g for 30 minutes, and the post-centrifugation supernatant was filtered through a 0.22 μm filter. The filtered stem cell medium supernatant was designated as test substance 4. Additionally, it was confirmed that the maintained iPS cells were positive for the undifferentiated markers NANOG, Oct3/4, and TRA-1-60.

Test substance 5 was prepared as follows. Human blood cell-derived iPS cells were established in a two-dimensional culture. After five passages, it was confirmed that the iPS cells contained no residual reprogramming factors and were free of viruses. Subsequently, the iPS cells were cryopreserved in a cryopreservation solution (STEM-CELLBANKER).

A 30 mL bioreactor was prepared, into which 20 mL of stem cell medium (Puel, I Peace, Inc.) was added. Single, undifferentiated iPS cells (4×10\hat{ }6 cells) frozen in STEM-CELLBANKER solution were seeded into the bioreactor, initiating three-dimensional agitation culture of the iPS cells. The medium was agitated at a rate allowing 20% to 60% of the clumps to settle.

Two days later, an additional 10 mL of stem cell medium was added to the bioreactor. Every two days thereafter, fresh stem cell medium was added until the formation of cell clusters. Flow cytometry analysis on the seventh day after seeding the iPS cells into the bioreactor confirmed positive expression of TRA1-60, a marker of pluripotent stem cells.

Next, the stem cell medium containing suspended iPS cells was filtered through a 0.22 μm mesh filter to remove cell clumps. The filtered medium was centrifuged at 1500 rpm for 5 minutes to pellet the cells, after which the supernatant was centrifuged again at 3000 rpm for 3 minutes. The resulting supernatant was then filtered through a 0.22 μm filter, with the filtered supernatant designated as test substance 5.

Test substance 6 was prepared as follows. The human iPS cells used to prepare test substance 4 were detached from the dish using TrypLE Select. The pelleted cells were then triturated using a pestle (Pestle in G-Tube, Thermo Fisher Scientific). The triturated human iPS cell paste was dissolved in DMEM/F12 to obtain a human iPS cell extract. This extract was stored at 4° C. for 1 to 24 hours, filtered, and frozen until use. Before being used in the following examples, the extract was thawed and designated as test substance 6.

Test substance 7 was prepared in the same manner as test substance 6, except that porcine blood cells were used to induce porcine iPS cells under the same conditions used for test substance 4. The resulting porcine iPS cell extract was designated as test substance 7.

As a positive control substance for Example 2, isoproterenol (Cayman), known to have lipolytic activity, was prepared. For Example 3, insulin (Thermo Fisher Scientific), known to induce differentiation from myoblasts to myotubes, was prepared as a positive control substance. For Example 4, arachidonic acid (Sigma-Aldrich, A3411), known to promote prostacyclin production, was prepared as a positive control substance. For Example 5, N-acetylglucosamine (Sigma-Aldrich, A8625), known to promote hyaluronic acid production, was prepared as a positive control substance.

### Example 2: Fat-Decomposing Assay

Normal human subcutaneous preadipocytes (Lonza, PT-5020) were seeded into a T-75 flask using a proliferation medium (PGM-2, 10% FBS, 2 mM L-glutamine, 50 μg/mL gentamicin, and 37 ng/mL amphotericin B-supplemented PBM, Lonza, PT-8002) and cultured in a CO₂ incubator (37° C., 5% CO₂, humidified) until they reached 60% to 80% confluence. The cells were then detached using 0.25% trypsin-EDTA and centrifuged. Subsequently, the cells were seeded into a 96-well plate at 10,000 cells/100 μL/well in proliferation medium and incubated in a CO₂ incubator.

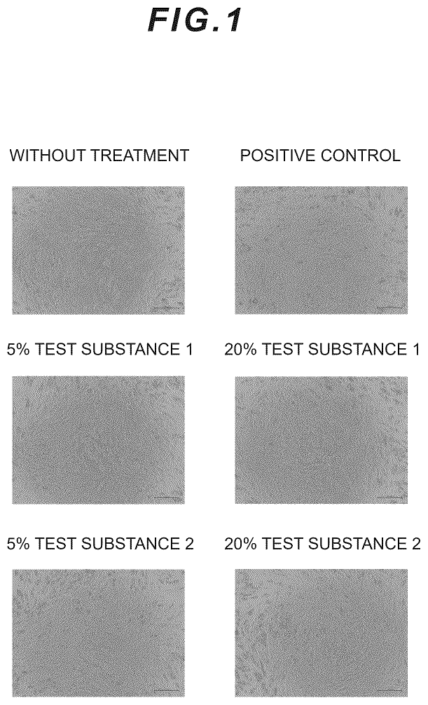

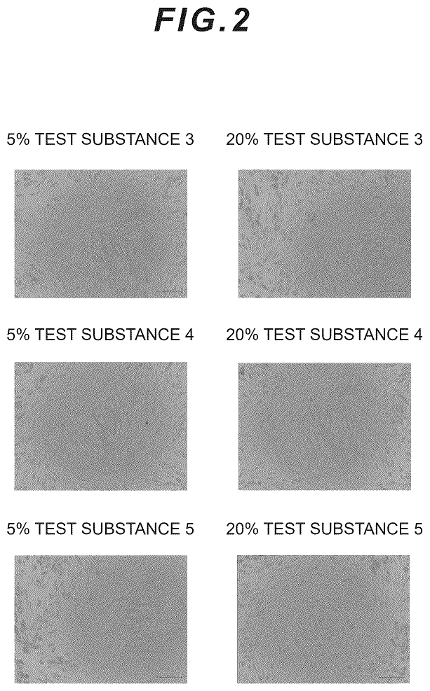

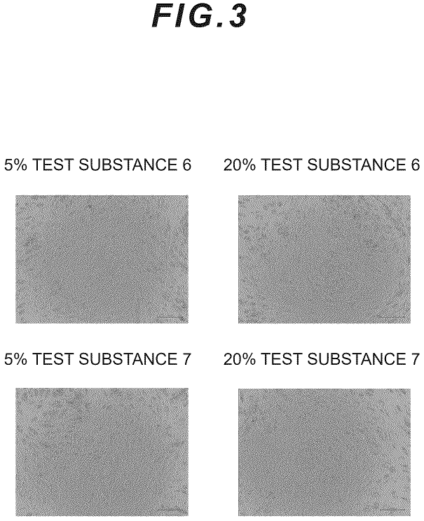

The following day, the medium was replaced with 100 μL of differentiation medium (PGM-2 supplemented with insulin, dexamethasone, indomethacin, and isobutyl-methylxanthine; Lonza, PT-8002), and the cells were cultured for seven days to induce differentiation of preadipocytes into adipocytes. The medium was then replaced with 100 μL of differentiation medium containing test substances 1 through 7 at final concentrations of 5% and 20%, or 10 μM positive control substance, or differentiation medium without test or control substances. The cells were cultured for 24 hours in a CO₂ incubator, and the adipocytes were treated with one of test substances 1 through 7 or the positive control. Images of the treated cells are shown in FIGS. 1, 2, and 3.

Thereafter, the culture supernatant was collected, and the amount of free glycerol in the supernatant was measured using a fat-decomposing assay kit (Cayman, 10009381). The amount of free glycerol corresponds to the degree of fat-decomposing. The measurement method followed the protocol provided with the kit. Specifically, a multi-spectral microplate reader (Varioskan Flash, Thermo Fisher Scientific) was used to measure the absorbance of the supernatant at a wavelength of 540 nm, and the relative concentration of free glycerol was calculated. The group without test substance addition was set as the control, and two-sided Dunnett's test was used to compare the control with the groups containing test substances and the positive control. A p-value of less than 0.05 was considered significant.

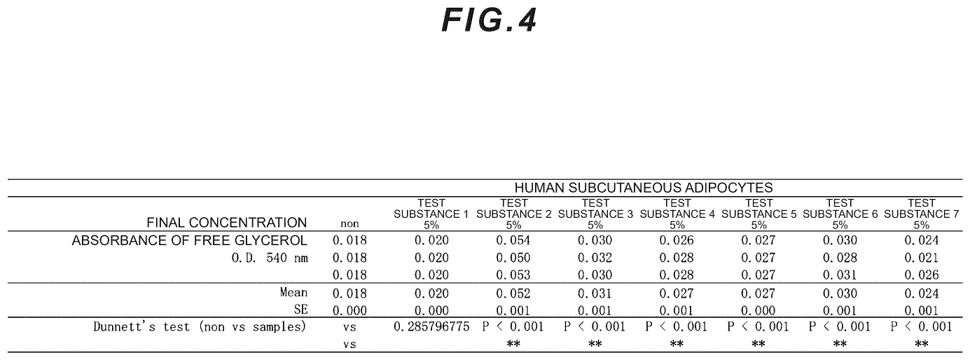

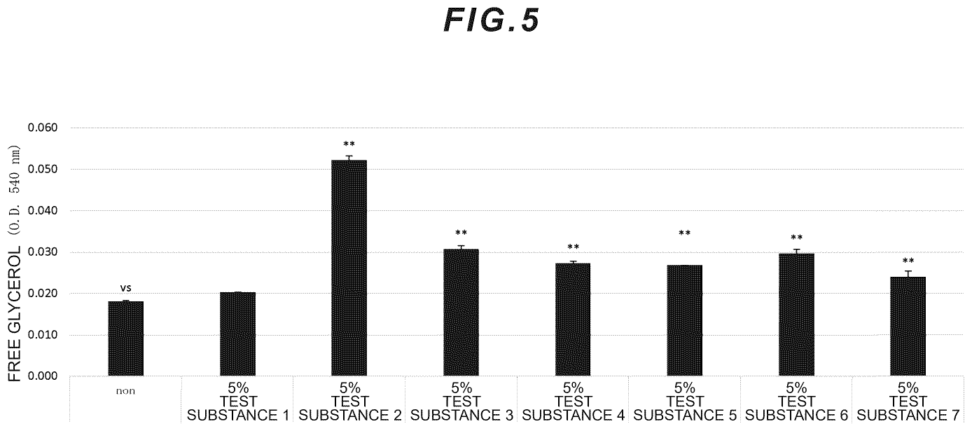

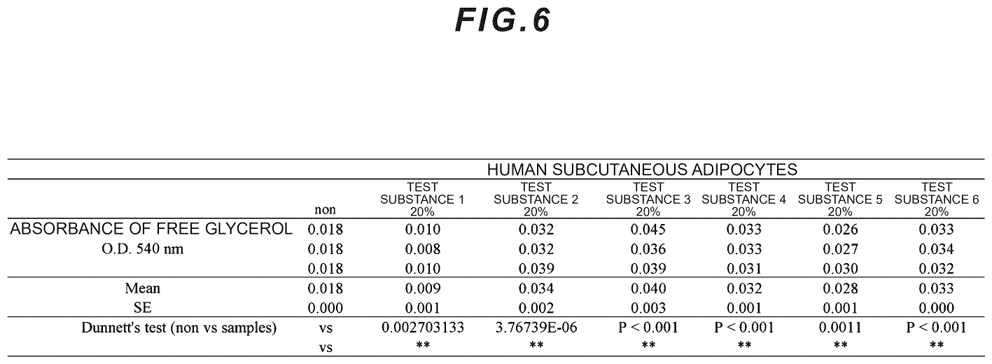

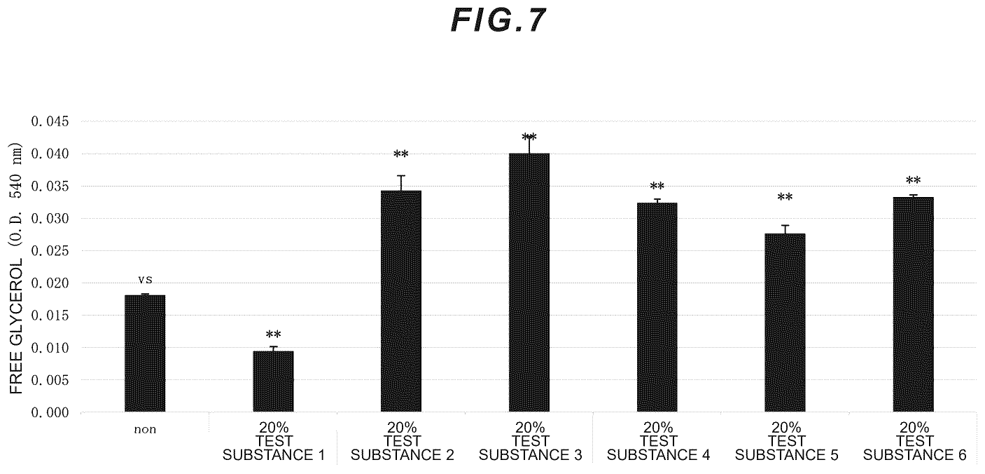

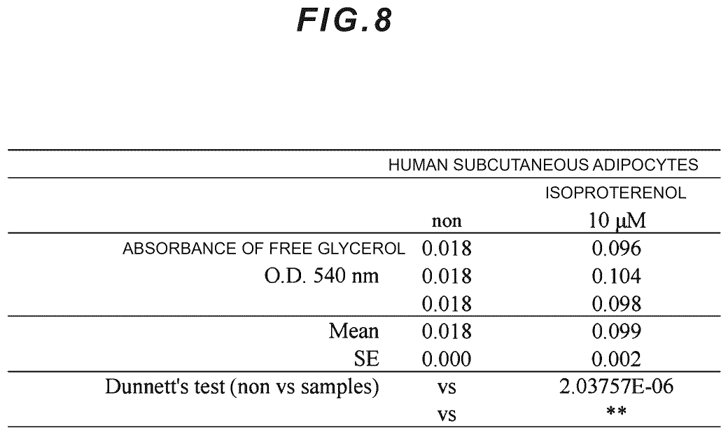

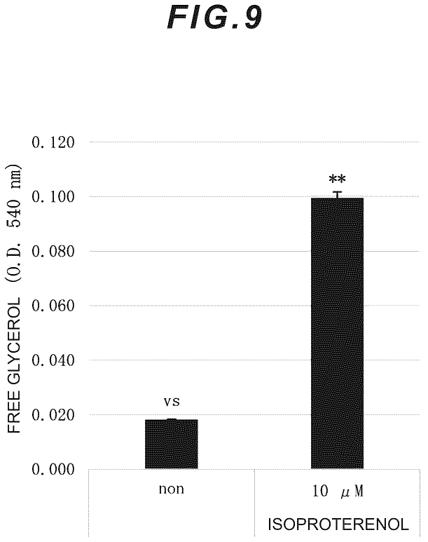

When the final concentration of test substances 1 through 7 was 5%, as shown in FIGS. 4 and 5, the groups to which test substances 2 through 7 were added showed significantly higher amount of fat-decomposition compared to the control. The group with test substance 1, which contained 5% DMEM, did not show a significant increase in the amount of fat-decomposition compared to the control. When the final concentration of test substances 1 through 6 was 20%, as shown in FIGS. 6 and 7, the groups with test substances 2 through 6 showed significantly higher amount of fat-decomposition compared to the control. The group with test substance 1, containing 20% DMEM, did not show a significant increase in the amount of fat-decomposition compared to the control. As shown in FIGS. 8 and 9, the group treated with isoproterenol, known to promote fat-decomposition, showed significantly higher amount of fat-decomposition compared to the control, indicating the accuracy of the experimental system. These results demonstrate that the stem cell medium, the medium used for induction culture of stem cells, the medium used for maintenance culture of stem cells, and the stem cell extract promote the fat-decomposition.

### Example 3: Myotube Differentiation Promotion Test

Using proliferation medium (SkGM-2, Lonza, CC-3245), normal human skeletal muscle myoblasts (Lonza, CC-2580) were seeded into a T-75 flask and cultured in a CO₂ incubator until approximately 60% confluence. The cells were then detached from the T-75 flask using 0.05% trypsin-EDTA and phenol red. The detached cells were seeded into a 96-well plate at 7,500 cells/100 μL/well in proliferation medium and cultured in a CO₂ incubator.

The next day, the medium was replaced with 100 μL of differentiation medium (2% horse serum-supplemented DMEM: F12) containing test substances 1 through 7 at final concentrations of 5% and 20% and the positive control substance at a final concentration of 50 nmol/L, or differentiation medium without test or positive control substances. The cells were then cultured for 3 days in a CO₂ incubator to induce differentiation of myoblasts into myotubes.

Subsequently, the medium was removed from the wells, and 100 μL of 4% paraformaldehyde at 4° C. was added to each well, followed by incubation at 4° C. for 15 minutes to fix the cells. The wells were then washed three times with 100 μL of Dulbecco's PBS, followed by the addition of 100 μL of blocking/permeabilization solution (3% bovine serum albumin and 0.3% Triton-X 100 in Dulbecco's PBS) for 30 minutes at room temperature.

Next, the solution in the wells was replaced with a solution containing an anti-myosin heavy chain (MHC) antibody (Affymetrix) and incubated overnight at 4° C. Myosin heavy chain serves as a differentiation marker for myotubes. After washing the wells three times with buffer, a secondary antibody (goat anti-mouse IgG2b, Alexa Fluor 555, Thermo Fisher Scientific) and a nuclear stain (Hoechst 33342, Thermo Fisher Scientific) were added, followed by incubation for 2 hours at room temperature. The wells were then washed three times with buffer, and buffer was added. Images were captured from nine fields of view in the center of each well using a confocal imaging system (Operetta CLS, PerkinElmer) with a 10× objective lens.

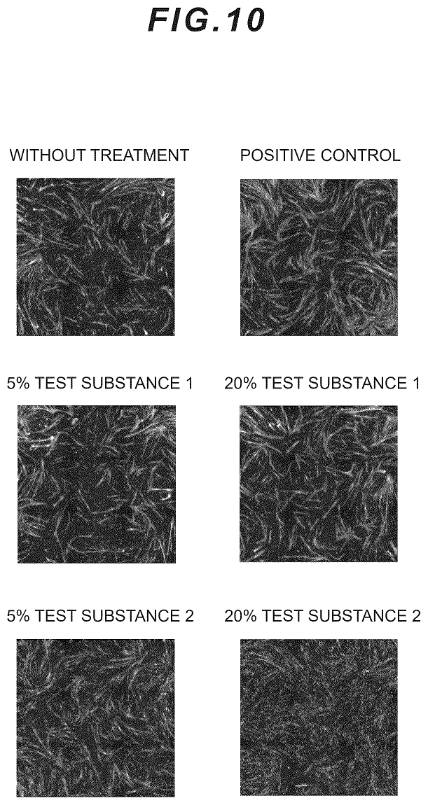

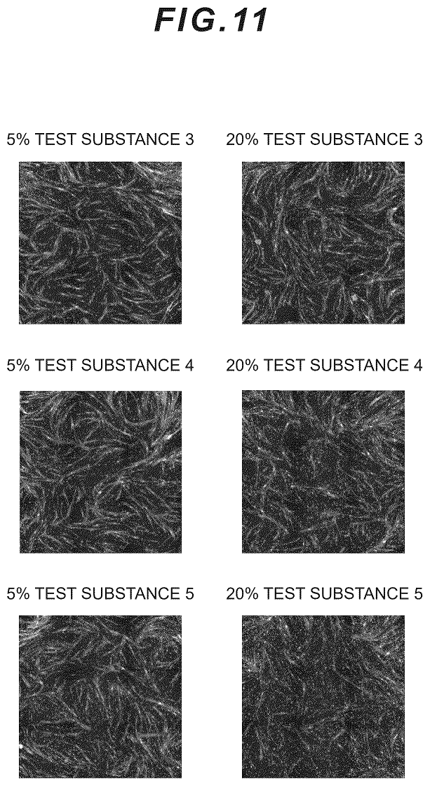

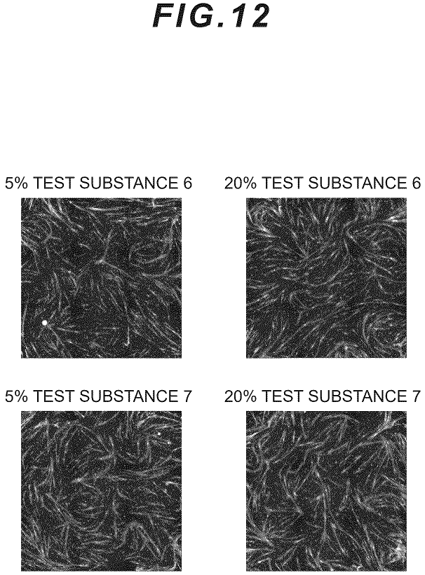

The images obtained through photography are shown in FIGS. 10, 11, and 12. From the captured images, the number of cell nuclei and the number of nuclei positive for myosin heavy chain were measured, and the differentiation index I (%) was calculated according to formula (1) below. A higher differentiation index I indicates that myotube differentiation has been more effectively promoted.

$$I = {N}_{M} / {N}_{T} \times 100 \qquad ( 1 )$$

In formula (1), N_{M} represents the number of nuclei positive for myosin heavy chain, and N_{T} represents the total number of nuclei.

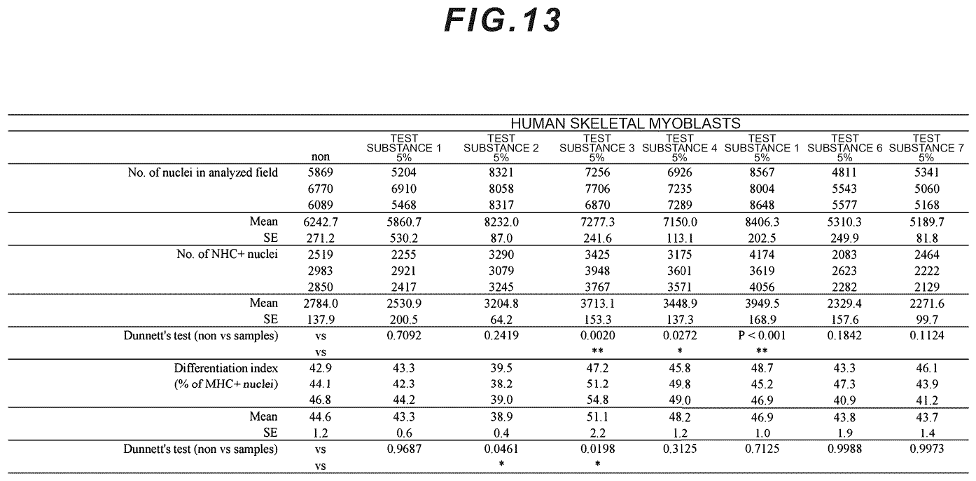

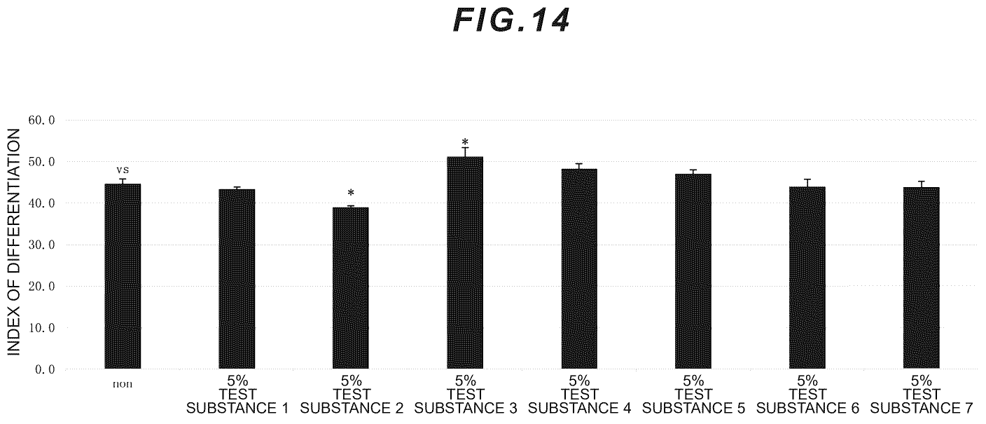

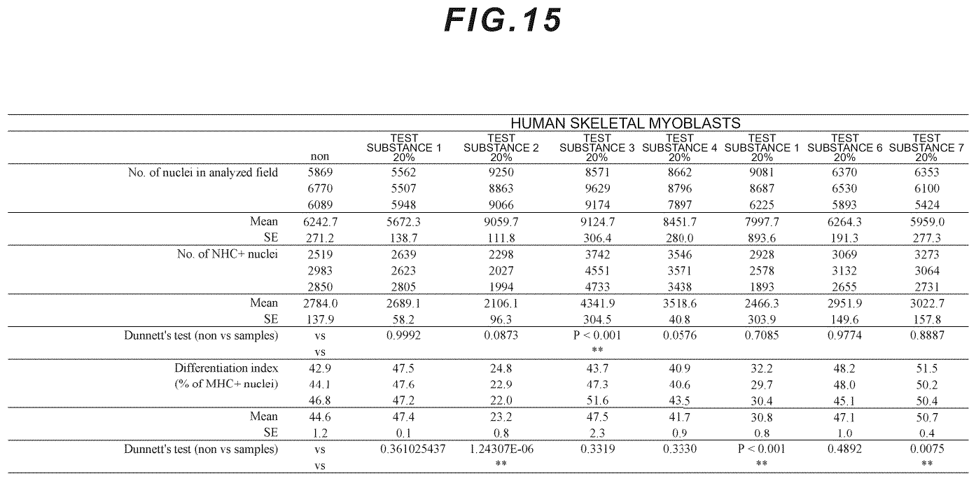

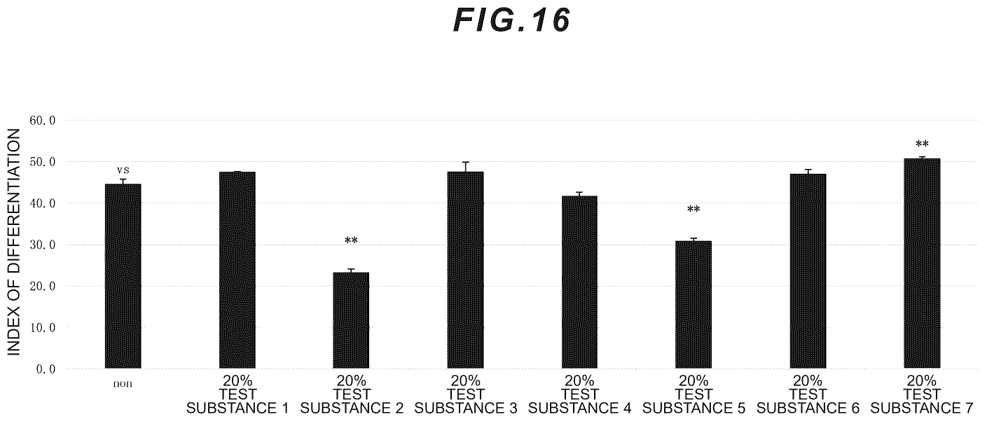

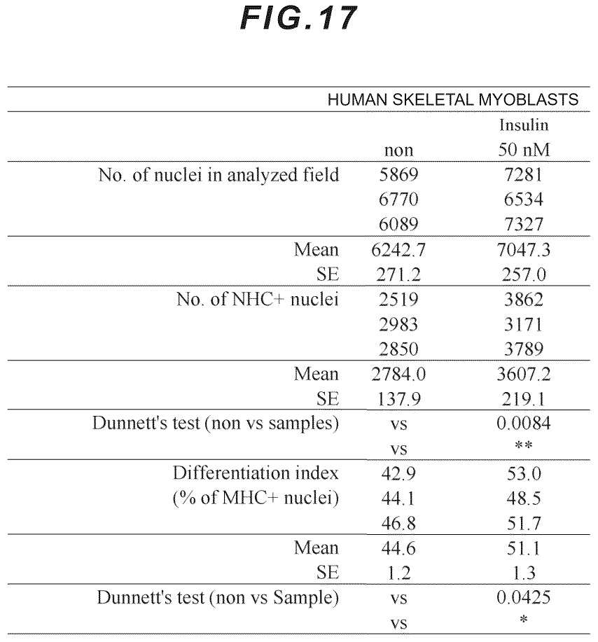

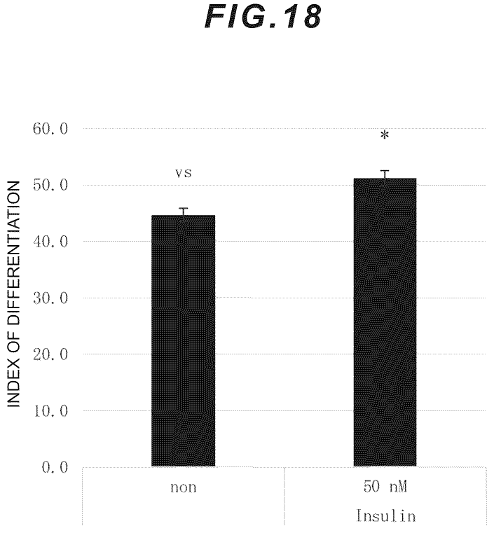

When the final concentration of test substances 1 through 7 was 5%, as shown in FIGS. 13 and 14, the groups with test substances 3 through 7 had significantly higher differentiation indices compared to the group with test substance 2, which contained only stem cell medium. When the final concentration of test substances 1 through 7 was 20%, as shown in FIGS. 15 and 16, the groups with test substances 3 through 7 had significantly higher differentiation indices compared to the group with test substance 2. As shown in FIGS. 17 and 18, the group treated with insulin, known to induce differentiation from myoblasts to myotubes, had a significantly higher differentiation index compared to the control, confirming the accuracy of the experimental system. These results demonstrate that the media used for induction and maintenance culture of stem cells and the stem cell extract promote differentiation of normal human skeletal muscle myoblasts into myotubes.

### Example 4: Prostacyclin Production Test

Normal human umbilical vein endothelial cells (Lonza, CC2519AS) were seeded into a T-75 flask using medium (EGM-2, Lonza, CC-3162) and cultured in a CO₂ incubator until 60% to 80% confluence. The cells were then detached from the T-75 flask using 0.05% trypsin-EDTA and phenol red. The detached cells were seeded into a 96-well plate at 10,000 cells/100 μL/well in medium and cultured in a CO₂ incubator.

The next day, the medium was replaced with 100 μL of medium containing test substances 1 through 7 at final concentrations of 5% and 20%, and the positive control substance at a final concentration of 10 μM, or medium without test or positive control substances. The cells were then cultured for 24 hours in a CO₂ incubator.

The culture supernatant was collected, and the amount of 6-keto prostaglandin Fla, a metabolite of prostacyclin, was measured using an ELISA kit (Abcam, ab133023) and a multi-spectral microplate reader (Varioskan Flash, Thermo Fisher Scientific).

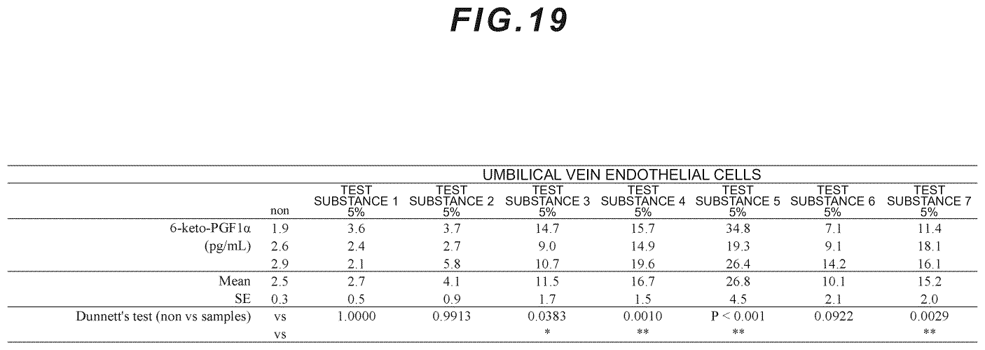

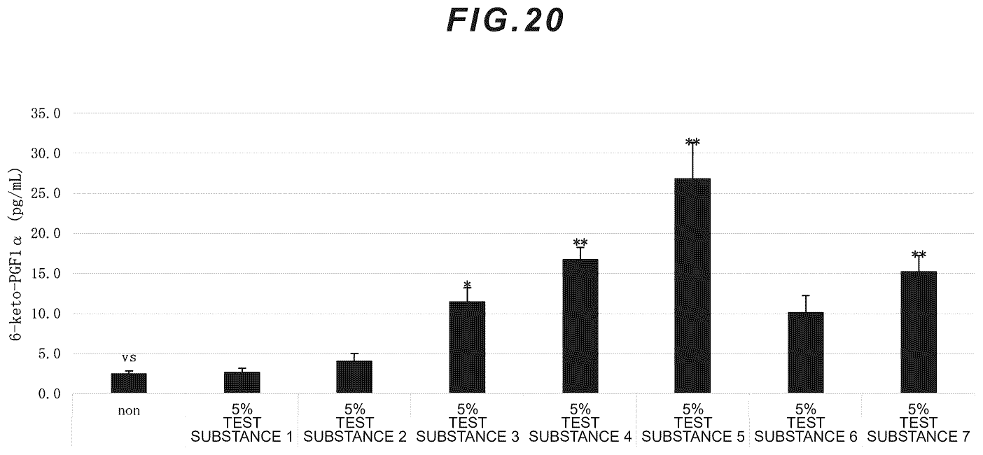

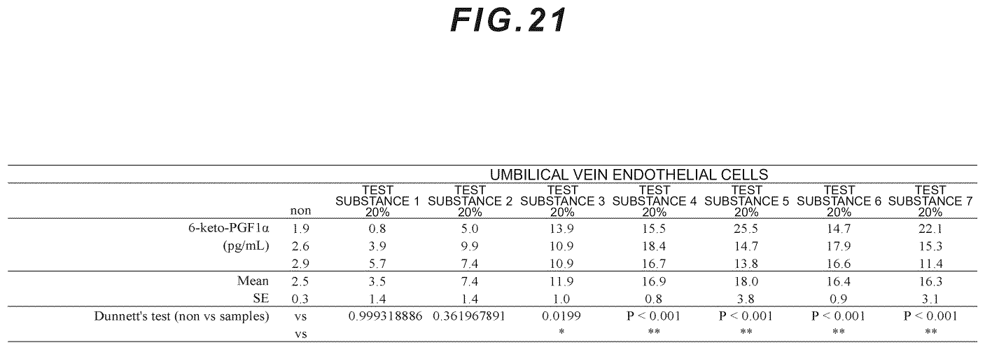

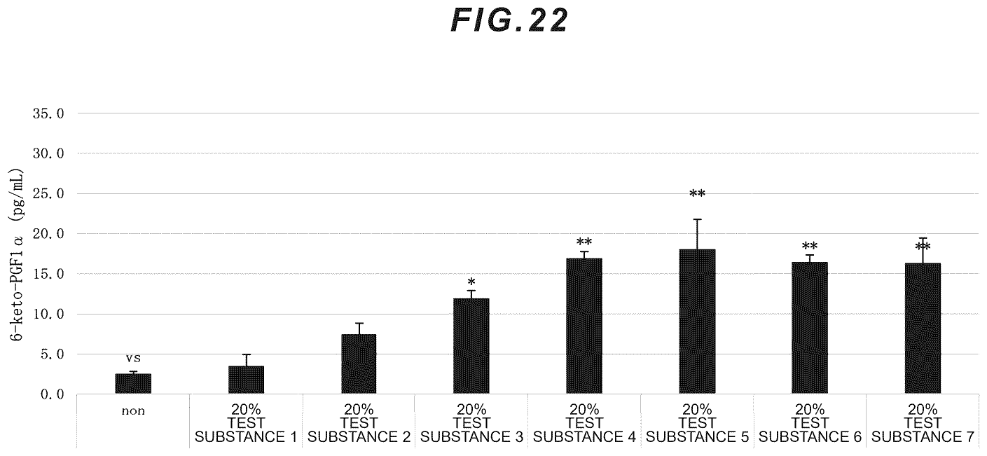

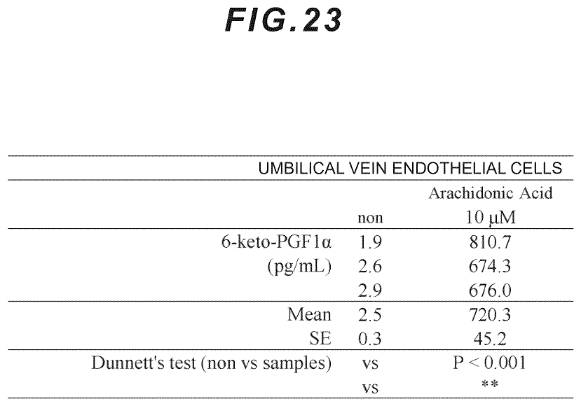

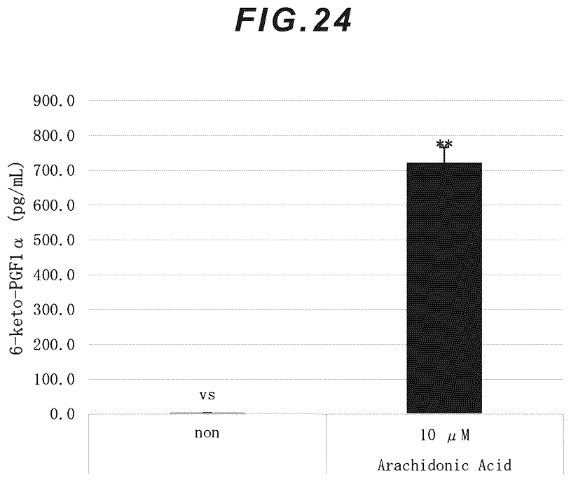

When the final concentration of test substances 1 through 7 was 5%, as shown in FIGS. 19 and 20, the groups with test substances 3 through 7 had significantly higher levels of 6-keto prostaglandin Fla compared to the control group without test substances. When the final concentration of test substances 1 through 7 was 20%, as shown in FIGS. 21 and 22, the groups with test substances 3 through 7 also had significantly higher levels of 6-keto prostaglandin Fla compared to the control. As shown in FIGS. 23 and 24, the group treated with arachidonic acid, known to promote prostacyclin production, showed significantly higher levels of 6-keto prostaglandin Fla compared to the control, verifying the accuracy of the experimental system. These results indicate that the media used for induction and maintenance culture of stem cells, as well as the stem cell extract, promote prostacyclin production by normal human umbilical vein endothelial cells.

### Example 5: Hyaluronic Acid Production Test

Using proliferation medium (DMEM/F-12 supplemented with 10% FBS and 1% penicillin-streptomycin mixture), a human chondrocyte cell line (SW1353, ATCC, HTB-94) was seeded into a T-75 flask and cultured in a CO₂ incubator until 60-80% confluency. The cells were then detached from the T-75 flask using 0.25% trypsin-EDTA and phenol red. Subsequently, using the proliferation medium, the human chondrocytes were seeded into a 96-well plate at a density of 10,000 cells/100 μL/well and cultured in a CO₂ incubator.

The following day, the medium was replaced with 100 μL of assay medium (DMEM/F-12 supplemented with 1% FBS and 1% penicillin-streptomycin mixture), containing each of test substances 1 through 7 at final concentrations of 5% and 20% or a positive control substance at a final concentration of 10 mmol/L, or assay medium without test substances or positive control. The human chondrocytes were cultured in a CO₂ incubator for 24 or 72 hours.

The culture supernatant was collected, and the amount of hyaluronic acid was measured using an ELISA kit (Hyaluronan Quantikine ELISA Kit, R&D Systems) and a multi-spectral microplate reader (Varioskan Flash, Thermo Fisher Scientific).

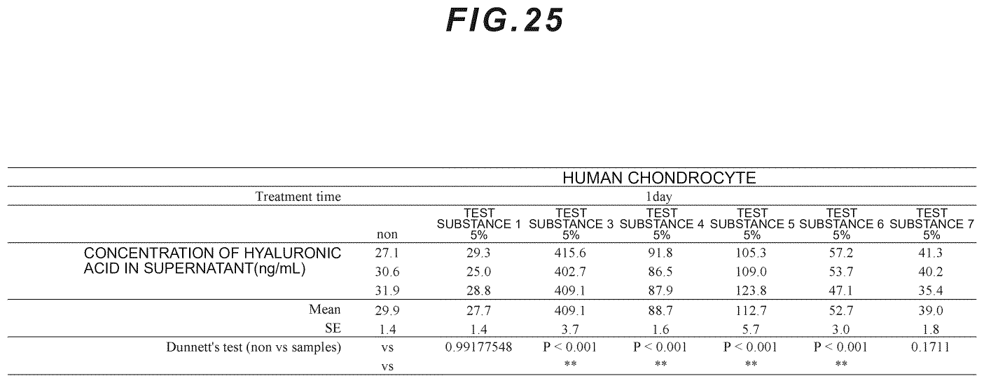

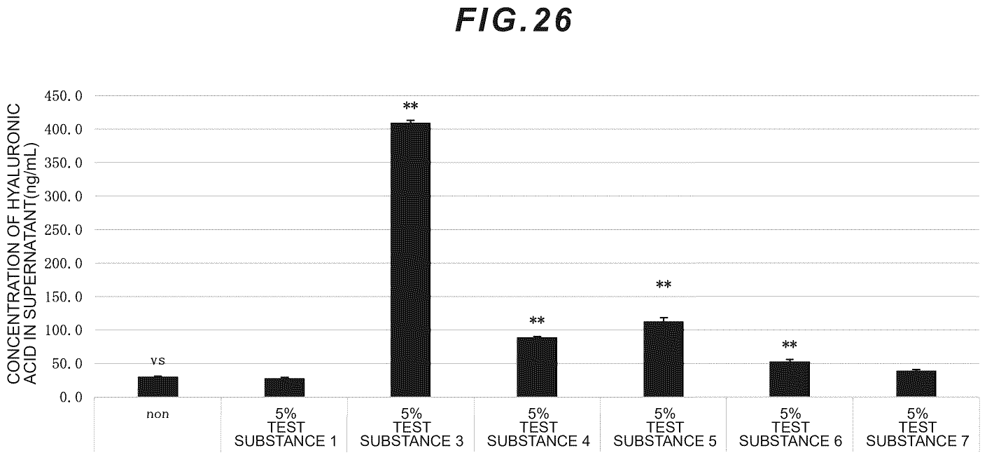

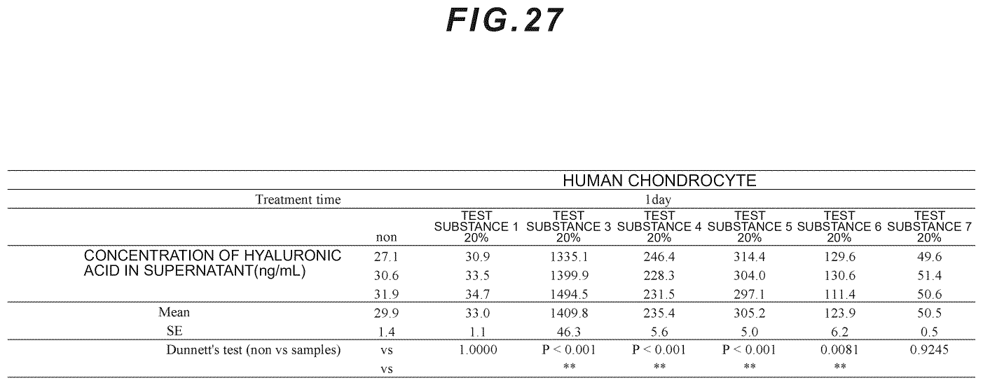

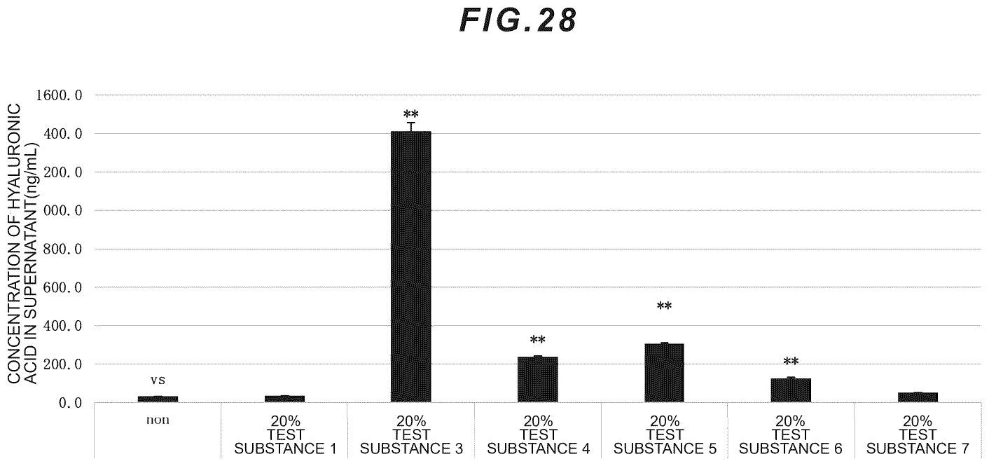

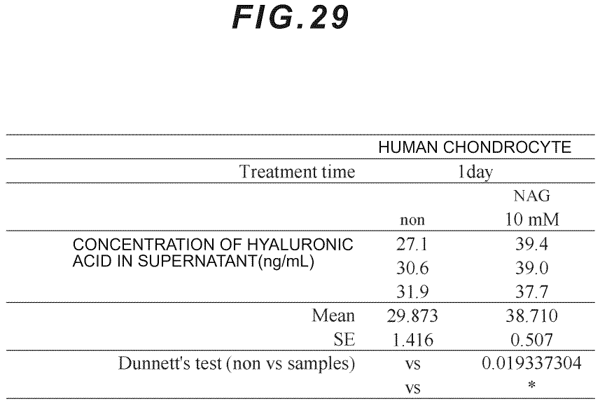

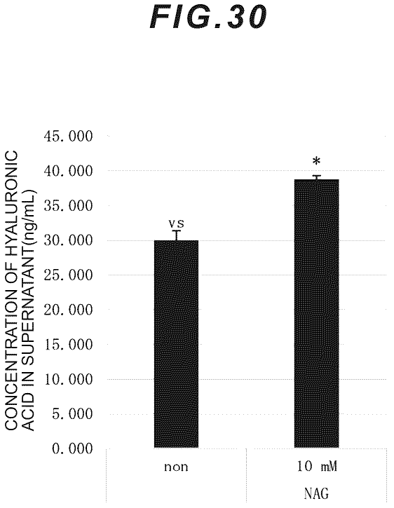

After culturing the human chondrocytes for 24 hours, when the final concentration of test substances 1 and 3 through 7 was 5%, as shown in FIGS. 25 and 26, the groups to which test substances 3 through 6 were added showed a significantly higher hyaluronic acid production compared to the control group without test substances. When the final concentration of test substances 1 and 3 through 7 was 20%, as shown in FIGS. 27 and 28, the groups with test substances 3 through 6 also showed significantly higher hyaluronic acid production compared to the control. As shown in FIGS. 29 and 30, the group with N-acetylglucosamine, known to promote hyaluronic acid production, showed significantly higher hyaluronic acid production compared to the control, confirming the validity of the experimental system. These results indicate that the medium used for the induction and maintenance culture of stem cells, as well as the stem cell extract, promote hyaluronic acid production by human chondrocytes.

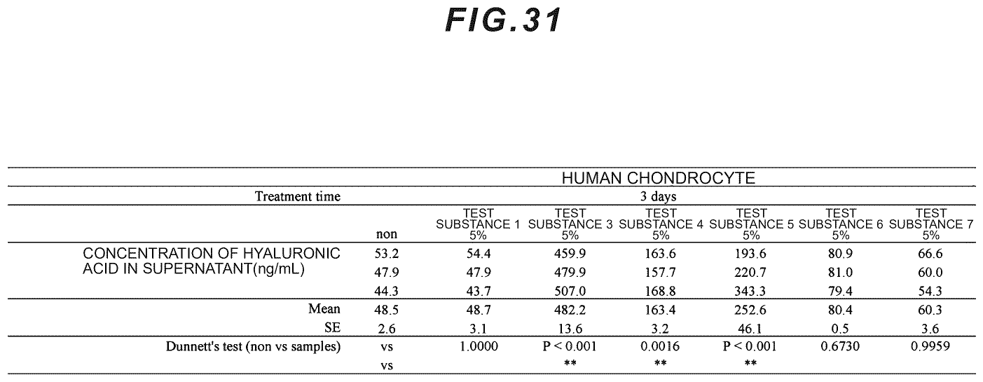

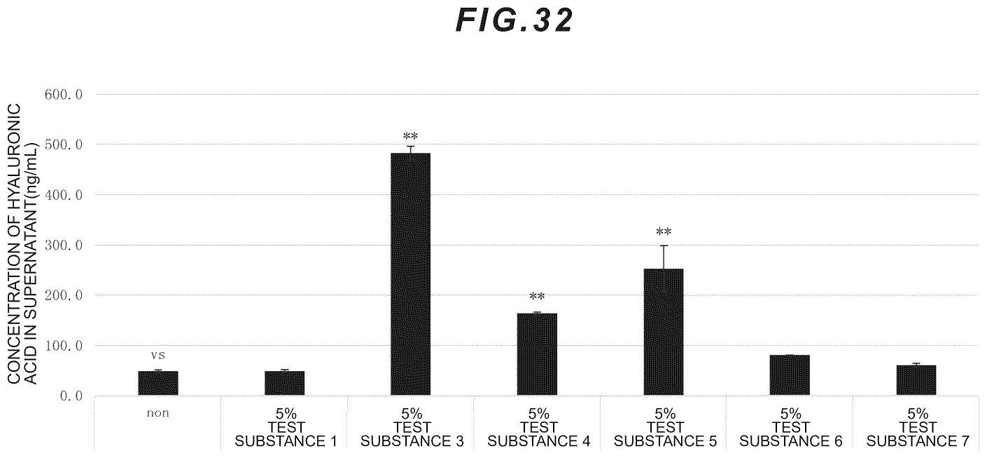

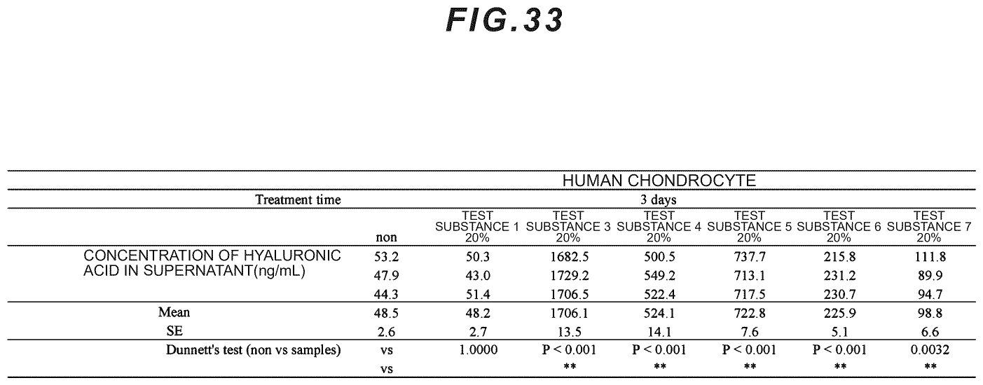

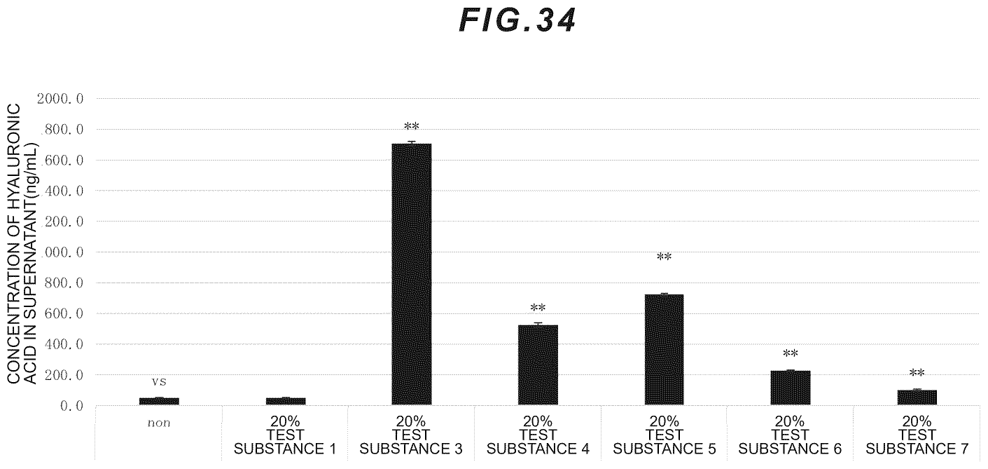

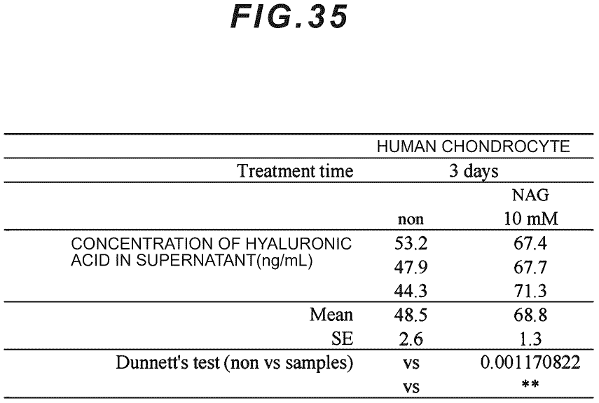

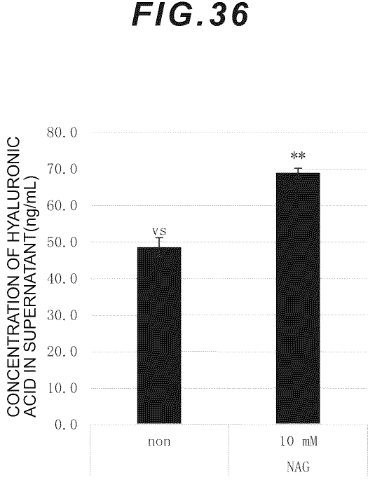

When human chondrocytes were cultured for 72 hours and the final concentration of test substances 1 and 3 through 7 was 5%, as shown in FIGS. 31 and 32, the groups with test substances 3 through 5 showed significantly higher hyaluronic acid production compared to the control group. When the final concentration of test substances 1 and 3 through 7 was 20%, as shown in FIGS. 33 and 34, the groups with test substances 3 through 7 had significantly higher hyaluronic acid production compared to the control. As shown in FIGS. 35 and 36, the group with N-acetylglucosamine, known to promote hyaluronic acid production, also had significantly higher hyaluronic acid production compared to the control, verifying the accuracy of the experimental system. These results demonstrate that the medium used for induction and maintenance culture of stem cells, as well as the stem cell extract, promotes hyaluronic acid production by human chondrocytes.

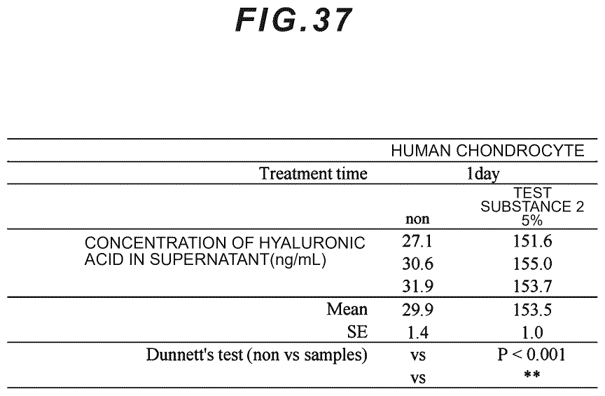

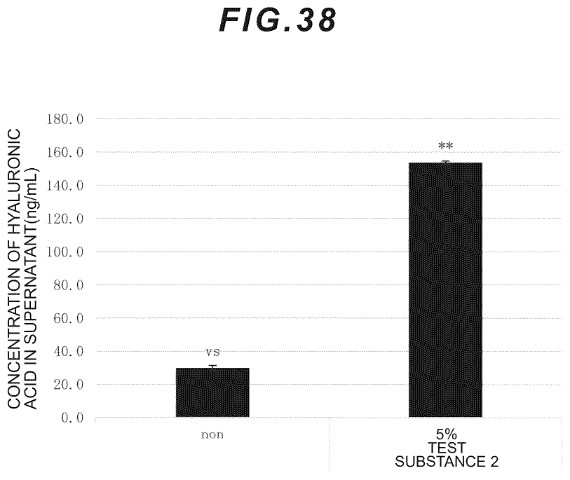

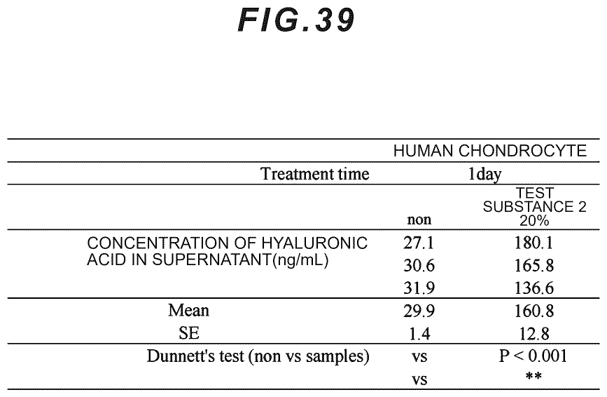

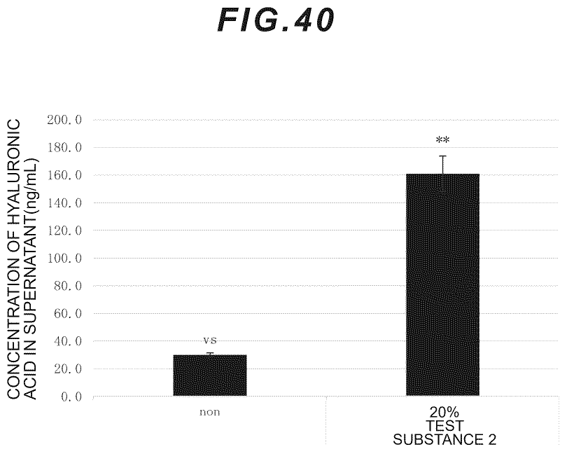

After culturing human chondrocytes for 24 hours with test substance 2 at a final concentration of 5%, as shown in FIGS. 37 and 38, the group with test substance 2 showed significantly higher hyaluronic acid production compared to the control. When human chondrocytes were cultured for 24 hours with test substance 2 at a final concentration of 20%, as shown in FIGS. 39 and 40, the group with test substance 2 also showed significantly higher hyaluronic acid production compared to the control. These results indicate that stem cell medium promotes hyaluronic acid production by human chondrocytes.

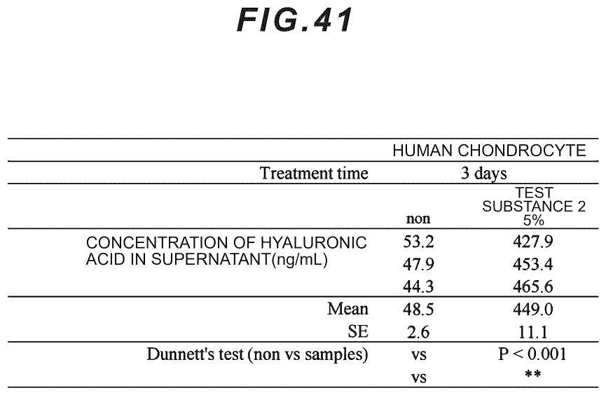

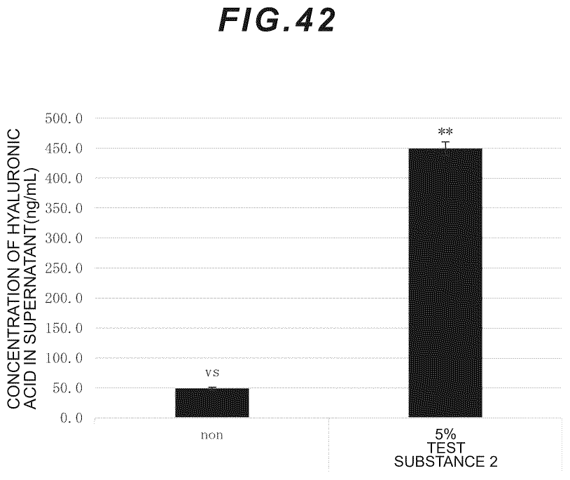

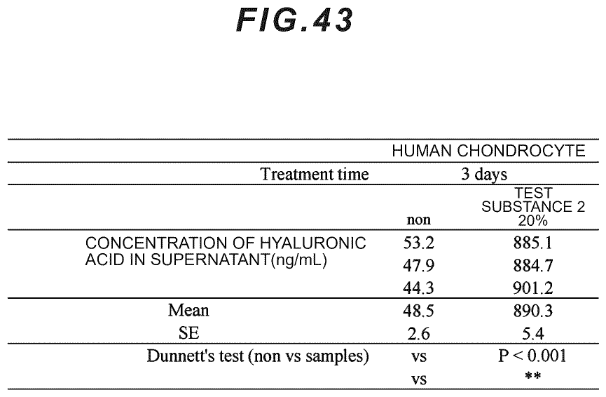

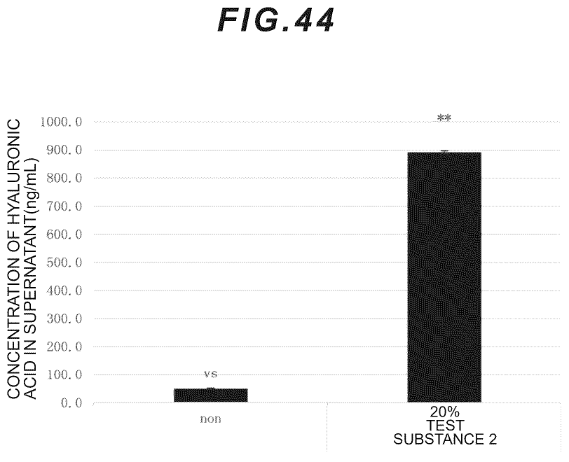

After culturing human chondrocytes for 72 hours with test substance 2 at a final concentration of 5%, as shown in FIGS. 41 and 42, the group with test substance 2 had significantly higher hyaluronic acid production compared to the control. When human chondrocytes were cultured for 72 hours with test substance 2 at a final concentration of 20%, as shown in FIGS. 43 and 44, the group with test substance 2 showed significantly higher hyaluronic acid production compared to the control.

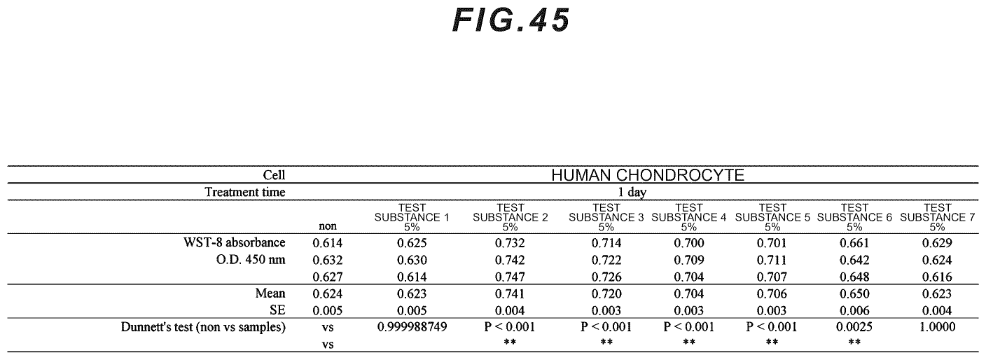

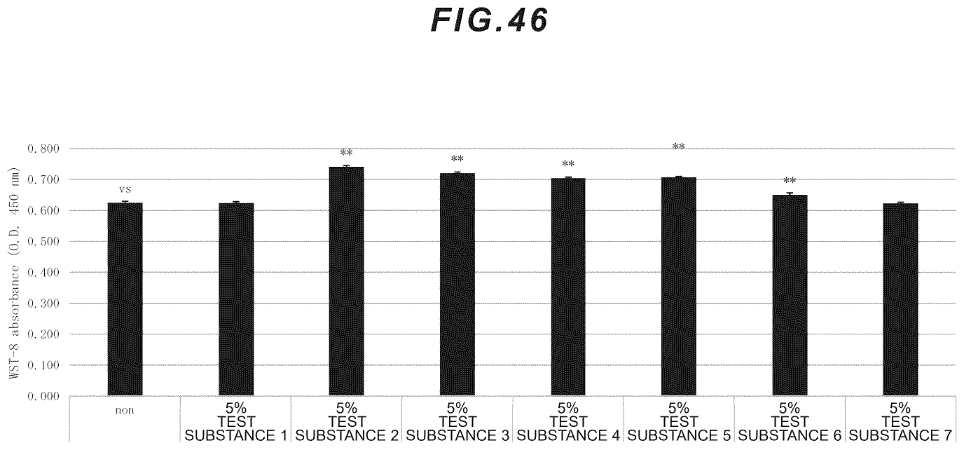

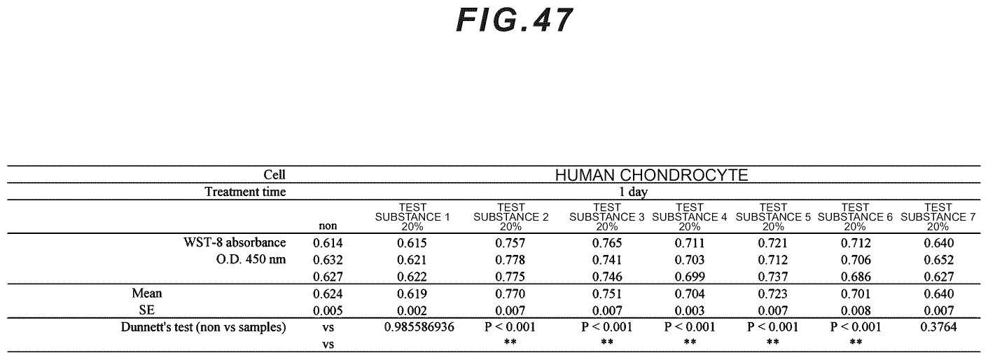

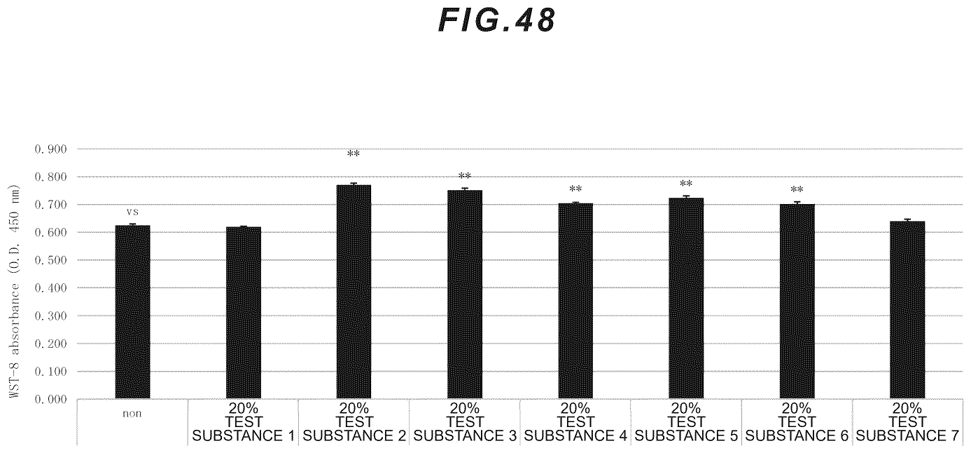

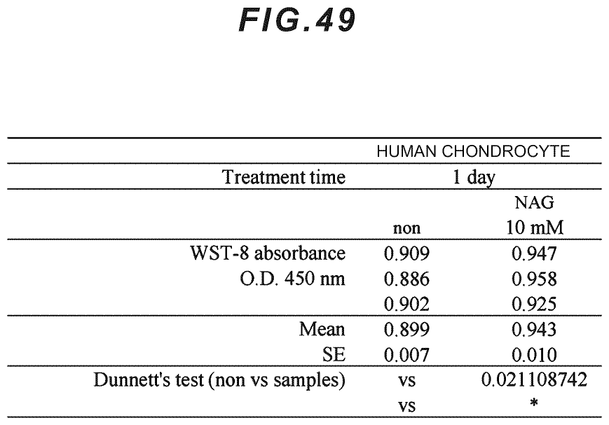

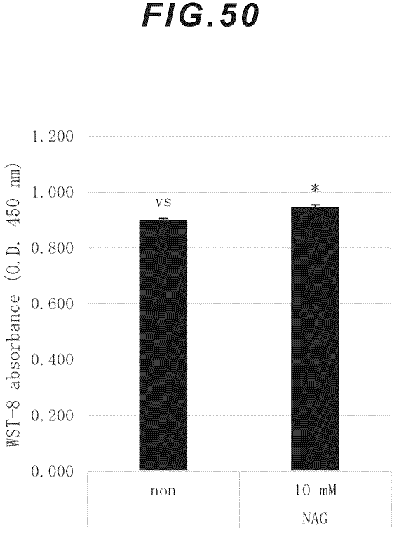

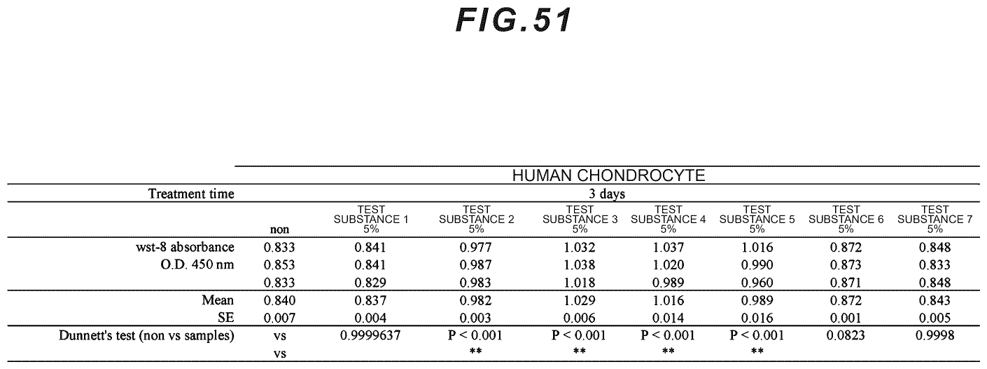

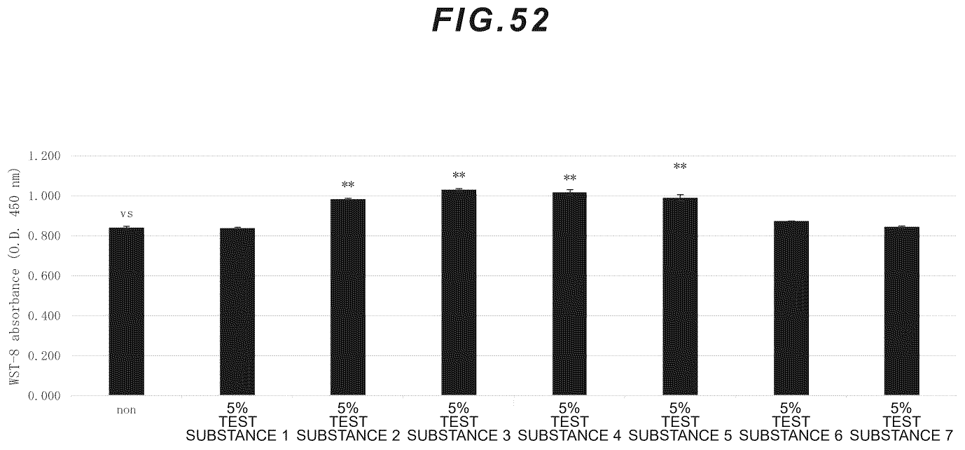

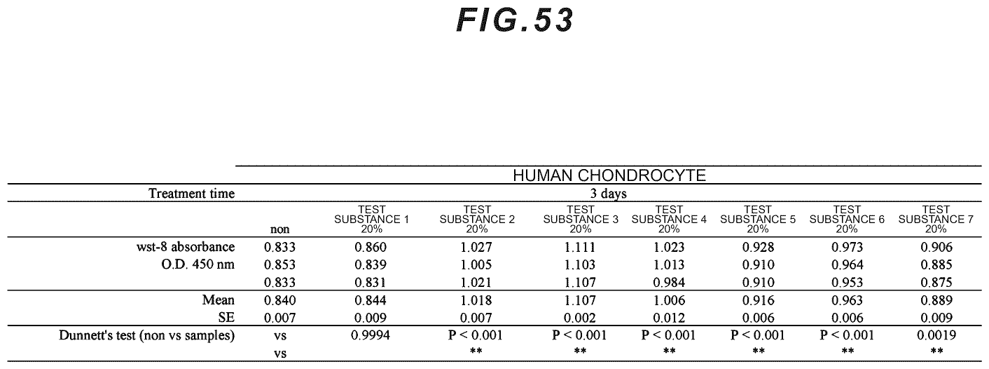

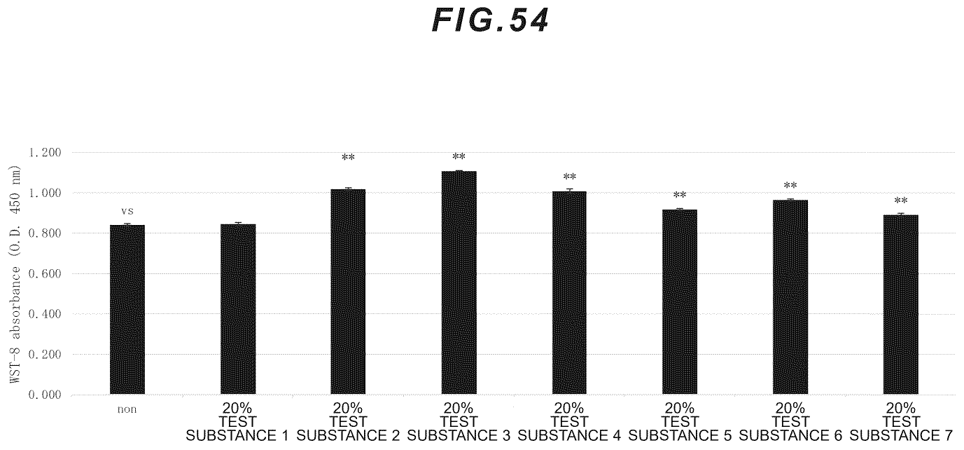

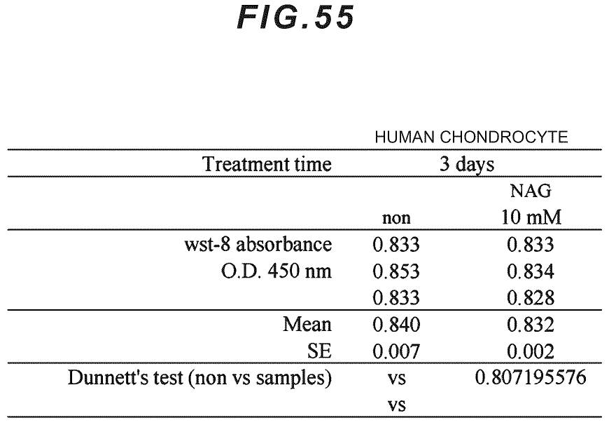

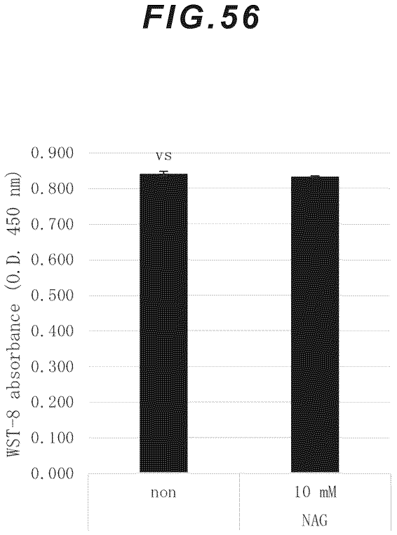

After collecting the culture supernatant, the cell viability was assessed using a WST-8 assay on the well plate. 100 μL of differentiation medium containing 10% Cell Counting Kit-8 (WST-8) reagent was added to each well, and the cells were incubated in a CO₂ incubator. The increase in absorbance at 450 nm per 60 minutes was measured with a multi-spectral microplate reader (Varioskan Flash, Thermo Fisher Scientific), and the relative absorbance was calculated as the relative cell viability. When human chondrocytes were cultured for 24 hours, as shown in FIGS. 45 to 50, the survival rate of human chondrocytes treated with test substances 2 through 7 was higher than that of test substance 1. These results indicate that the medium used for induction and maintenance culture of stem cells, as well as the stem cell extract, increases cell viability and promotes the proliferation of human chondrocytes. Similarly, when human chondrocytes were cultured for 72 hours, as shown in FIGS. 51 to 56, the survival rate of human chondrocytes treated with test substances 2 through 7 was higher than that of test substance 1, indicating that the medium used for the induction and maintenance culture of stem cells, as well as the stem cell extract, activates human chondrocytes, enhances cell viability, promotes proliferation, and rejuvenates human chondrocytes.

### Reference Example

Human iPS cells were cultured according to the examples described in JP 2016-128396. Specifically, using the same stem cell medium as in Example 1, human iPS cells were cultured in adhesion maintenance on feeder cells in adhesive culture dishes. The human iPS cells were passaged weekly. During passage, the human iPS cells were treated with a detachment solution containing 0.25% trypsin, 0.1 mg/mL collagenase IV, 1 mmol/L CaCl₂, and 20% KSR.

The human iPS cells cultured as described above were detached from the adhesion culture dish using an ES cell dissociation solution (TrypLE Select, trademark, Thermo Fisher Scientific). The detached human iPS cells were then suspended and cultured in a non-adherent culture dish for one week in ungellified iPS medium, resulting in the formation of embryoid bodies (EBs). The formed embryoid bodies were seeded onto an adhesive culture dish and allowed to grow for one week in DMEM containing 10% FBS and 1% Anti-Anti (antifungal agent).

Subsequently, the cells were detached from the adhesive culture dish using 0.05% trypsin-EDTA solution, and the single-cell suspension was seeded onto a new adhesive culture dish. The cells were then cultured for one week using DMEM containing 10% FBS as the medium.

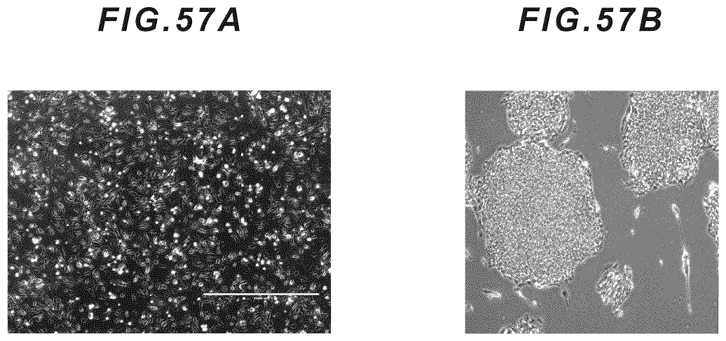

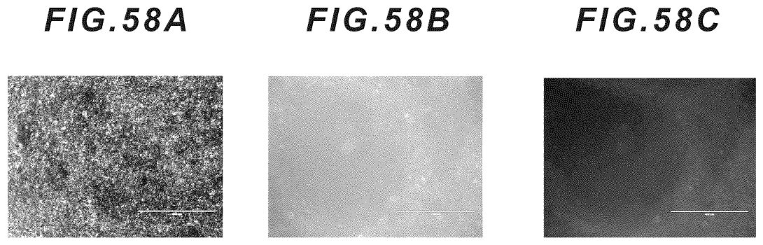

After confirming that the cells had reached 70-80% confluency, they were observed microscopically. A photograph of the cells cultured in this reference example is shown in FIG. 57A. Typically, the morphology of undifferentiated iPS cells appears as shown in the photograph in FIG. 57B. Thus, the cells cultured in this reference example were morphologically observed to not retain their undifferentiated state. Additionally, after culturing the cells for 21 days, the cells were treated with an anti-OCT3/4 antibody and an anti-NANOG antibody, each labeled with a fluorescent reagent, and observed under a microscope. The results are shown in FIG. 58. FIG. 58A shows a photograph of the cells observed without excitation light. FIG. 58B shows a photograph of the cells observed under excitation light corresponding to the fluorescent reagent bound to the anti-OCT3/4 antibody, and FIG. 58C shows a photograph of the cells observed under excitation light for the anti-NANOG antibody. No fluorescence was observed in FIGS. 58B and 58C, confirming that the cells were negative for OCT3/4 and NANOG. Furthermore, flow cytometry analysis confirmed that the cultured cells were negative for the undifferentiated markers NANOG, OCT3/4, and TRA-1-60, indicating that the cells did not maintain their undifferentiated state and had undergone differentiation.

### Example 6: Treatment Test in Osteoarthritis Model Rats

Male rats, five weeks of age, were anesthetized via isoflurane inhalation. Using a 30G injection needle and a microsyringe, 50 μL of 20 mg/mL monoiodoacetate (MIA) was injected into the left knee joint cavity to induce an osteoarthritis model in the left hind limb. Three days post-MIA injection, the rats were divided into a test group and a control group, with six rats in each group, ensuring no significant difference in the average left hind limb load-bearing ratio between the two groups.

The left hind limb load-bearing ratio was measured as follows: Each rat was placed in an incapacitance test apparatus (Linton Instrumentation), and while in a stable, relaxed state, the load on the left and right hind limbs was measured three consecutive times. The left hind limb load-bearing ratio, R (%), was calculated using the formula below:

$$R = L / ( L + R ) * 100 \qquad ( 2 )$$

where L is the average load on the left hind limb and R is the average load on the right hind limb.

On days 3, 7, 11, 15, and 19 after MIA injection, each rat in the test group was anesthetized via isoflurane inhalation, and 50 μL of Test Substance 4 was injected into the left knee joint cavity using a 30G injection needle and a microsyringe. In the control group, each rat received 50 μL of saline in the same manner on day 3 post-MIA injection.

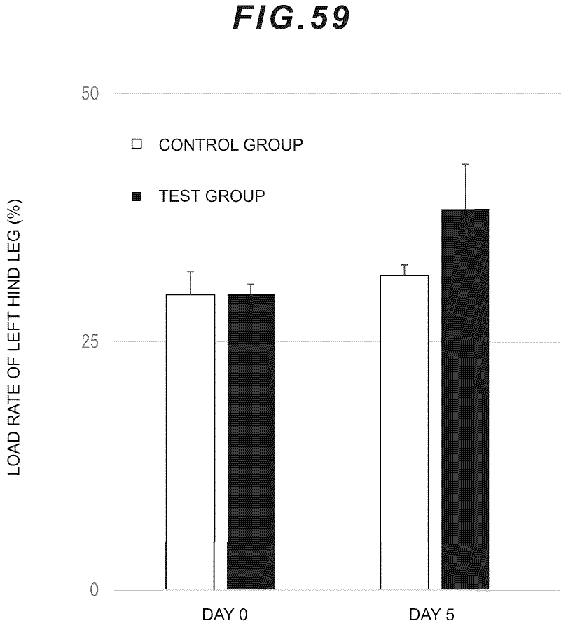

On day 5 after MIA injection, the left hind limb load-bearing ratio was measured for both the test and control groups using the same method. As shown in FIG. 59, the left hind limb load-bearing ratio was higher in the test group rats administered Test Substance 4 compared to the control group rats, indicating that Test Substance 4 alleviated osteoarthritis in the left hind limb and reduced joint pain. Upon dissection of the rats' joints, an increased number of chondrocytes and substantial cartilage tissue were observed in the joints administered Test Substance 4, demonstrating the therapeutic effect of Test Substance 4 on joint disease. These findings indicate that Test Substance 4 improved the survival and proliferation rates of chondrocytes and enhanced hyaluronic acid production by chondrocytes.

### Example 7: Preparation of the Supernatant from the Medium Used for Maintaining iPS Cells

Human iPS cells (5×10⁵ cells) were seeded onto a laminin-coated 10 cm dish and cultured in Puel medium for five days until they reached 80% confluency, with the medium changed every two days. On the fifth day after seeding, the medium was replaced with fresh Puel medium (10 mL), and on the seventh day, the culture supernatant was collected in a 15 mL tube. The supernatant in the 15 mL tube was centrifuged at 200 g for 5 minutes, filtered using a 0.22 μm syringe filter, and the supernatant obtained was designated as the culture supernatant in Example 7.

### Example 8: In Vitro Expression of Rejuvenation Genes

A six-well plate was prepared. Human dermal fibroblasts (1×10⁵ cells) were seeded in two wells and cultured in skin cell medium. The following day, the skin cell medium was completely removed from each well. For the first well, 2 mL of the supernatant from the culture medium in Example 7 was added, while the second well, serving as the control, was treated with skin cell medium. After two days, the skin cells were detached using trypsin, collected into a 1.5 mL tube, and centrifuged, and the supernatant was discarded.

RNA was extracted from the skin cells using the Total RNA Purification Kit (NucleoSpin RNA Plus, MACHEREY-NAGEL). Using a cDNA synthesis kit for real-time PCR (ReverTra Ace qPCR RT Master Mix, TOYOBO), cDNA was synthesized from RNA, followed by qPCR with a real-time PCR master mix (THUNDERBIRD Next SYBR qPCR Mix, TOYOBO). Gene expression levels of GDF11, MANF, TIMP2, SPARCL1, THBS4, GAPDH, NAMPT2, NANOG, TERT, p53, and p16 in the skin cells were analyzed. Each gene expression level was normalized to GAPDH expression.

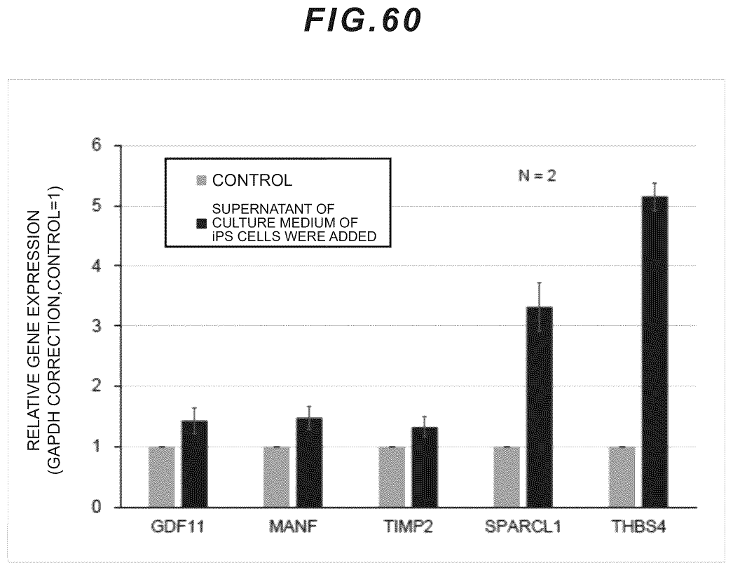

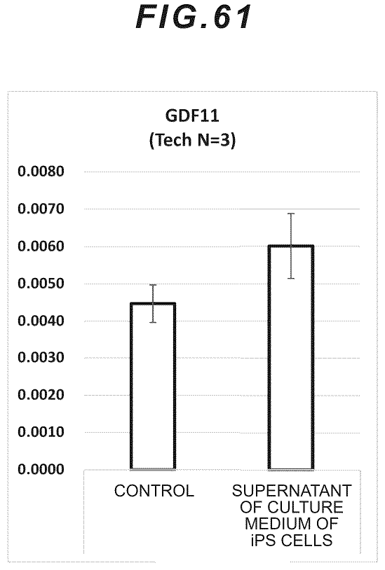

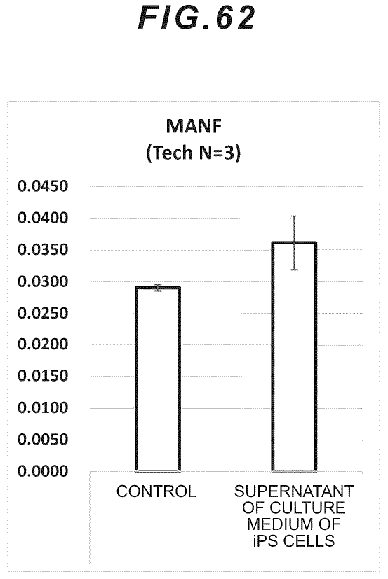

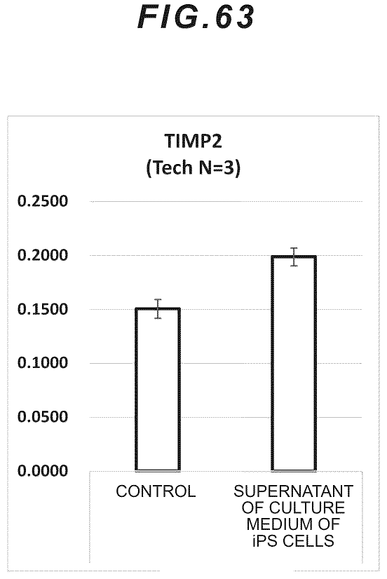

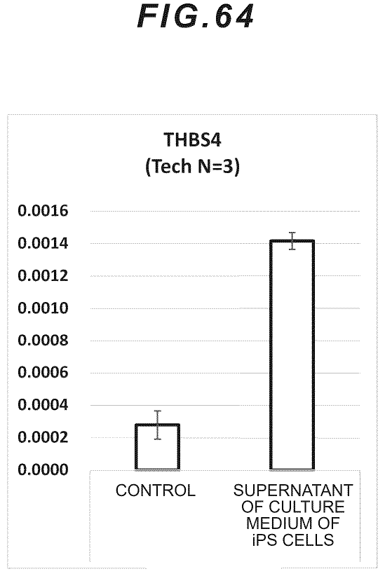

As shown in FIGS. 60, 61, 62, 63, and 64, the addition of the culture supernatant from Example 7 to the skin cells resulted in an increase in the expression of GDF11, MANF, TIMP2, SPARCL1, and THBS4. These genes are known to decrease throughout the body as aging progresses. The observed increase in the expression of GDF11, MANF, TIMP2, SPARCL1, and THBS4 indicates that the skin cells exhibited rejuvenation upon addition of the culture supernatant, suggesting an anti-aging effect of the culture supernatant.

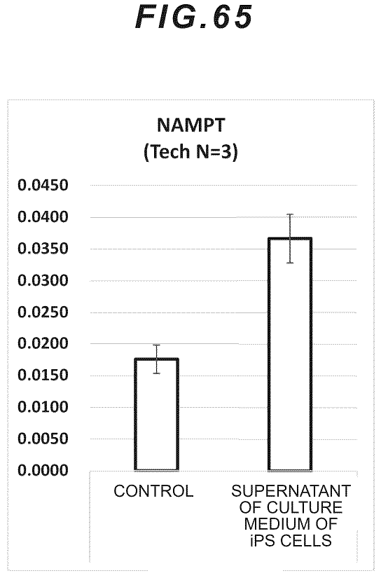

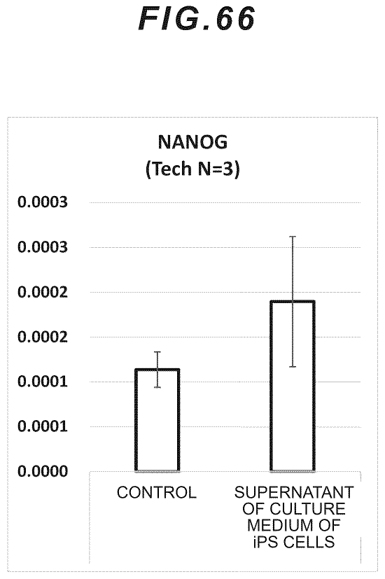

Additionally, as shown in FIGS. 65 and 66, the addition of the culture supernatant from Example 7 to skin cells increased the expression of NAMPT2 and NANOG. These genes are known to decrease throughout the body with aging. The increase in NAMPT2 and NANOG expression suggests that the skin cells exhibited signs of rejuvenation. NANOG is known as a reprogramming factor, and its increased expression indicates that adding the culture supernatant induced cellular reprogramming and rejuvenation, demonstrating the anti-aging potential of the culture supernatant.

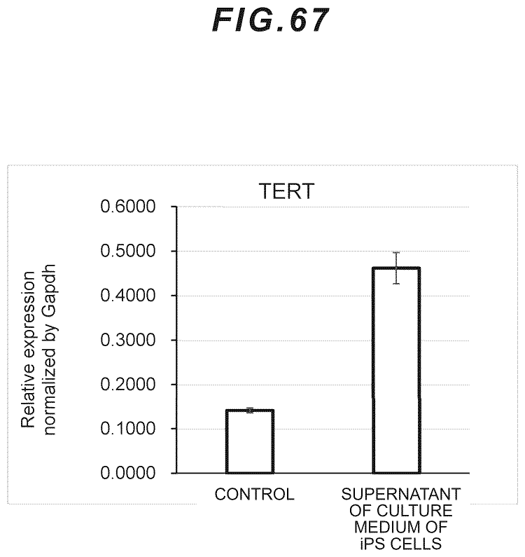

Furthermore, as shown in FIG. 67, the addition of the culture supernatant from Example 7 to the skin cells resulted in an increase in TERT expression. TERT enhances the expression of telomere elongation enzymes. This result indicates that the culture supernatant may be used as a telomere-extending agent and telomere-shortening suppressor.

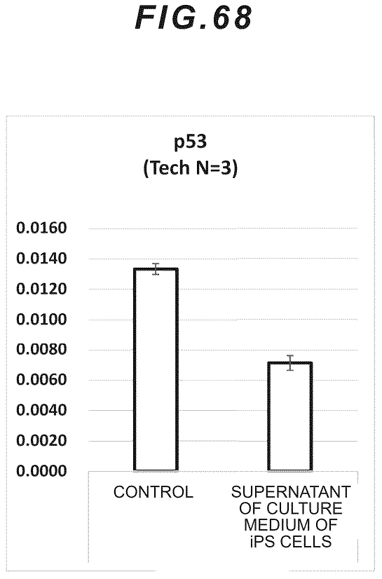

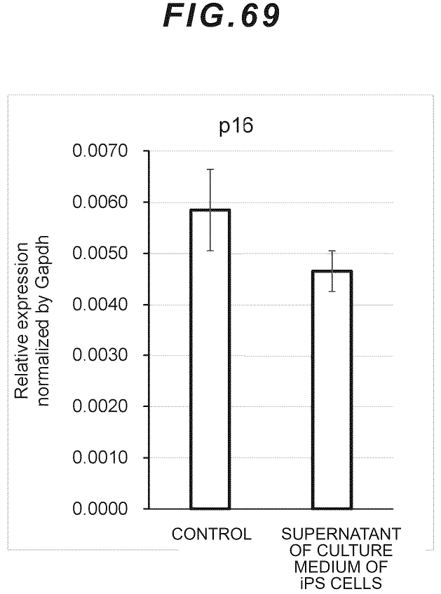

As shown in FIGS. 68 and 69, the addition of the supernatant from the culture medium in Example 7 to skin cells resulted in a decrease in the expression levels of p53 and p16. Both p53 and p16 are recognized as markers of cellular senescence, with their expression levels known to increase as cells undergo division. The reduction in p53 and p16 expression indicates that the addition of the culture medium supernatant induced a rejuvenating effect in the skin cells. Furthermore, this suggests that the culture supernatant exhibits anti-senescence and anti-cellular aging properties.

### Example 9: In Vivo Expression of Rejuvenation Genes

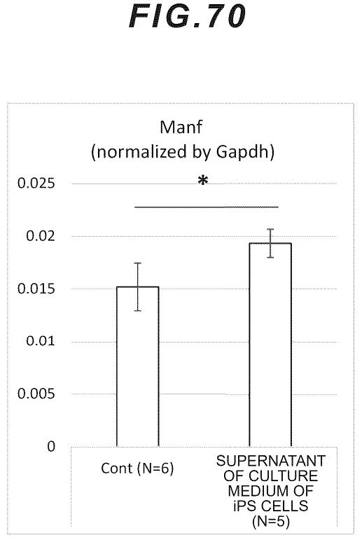

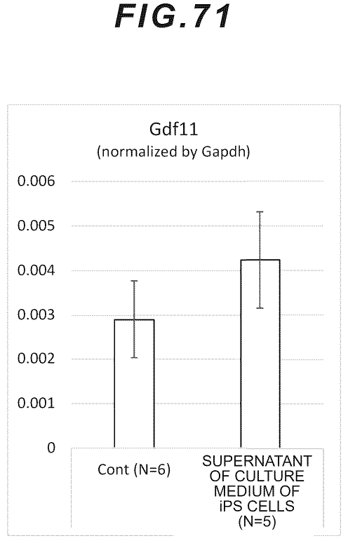

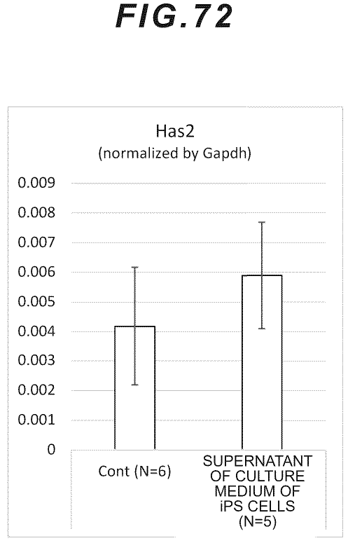

A total of 11 rats (6 male and 5 female), each aged six weeks, were prepared. For 14 days, each male rat received an injection of 5 mL of the culture medium supernatant from Example 7 per kilogram of body weight via the tail vein, administered once daily. Similarly, 5 mL of saline per kilogram of body weight was injected daily into the tail veins of a control group consisting of 6 male and 5 female rats. Two weeks after the initial injection, fibroblasts were isolated from each rat, and the expression levels of Manf, Gdf11, and Has2 in these fibroblasts were analyzed. As shown in FIGS. 70, 71, and 72, the expression levels of aging-suppressing factors such as Manf, Gdf11, and Has2 were elevated in the skin tissues of rats intravenously administered with the culture supernatant, as compared to the control group. This indicates that the rats exhibited delayed aging and a rejuvenating effect.

### Example 10: Expression of Hyaluronic Acid-Related Genes

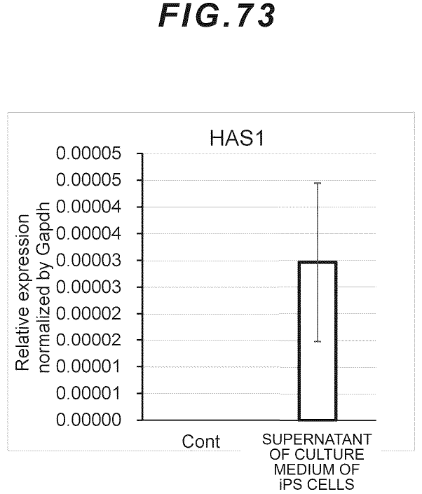

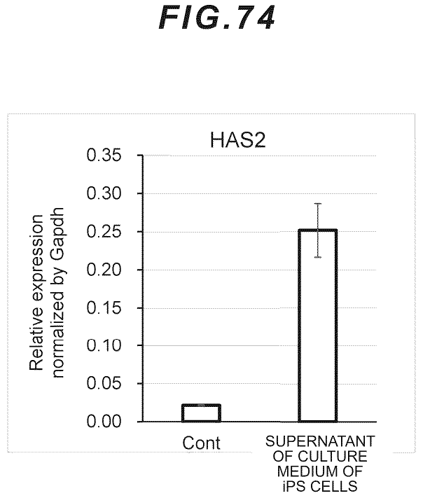

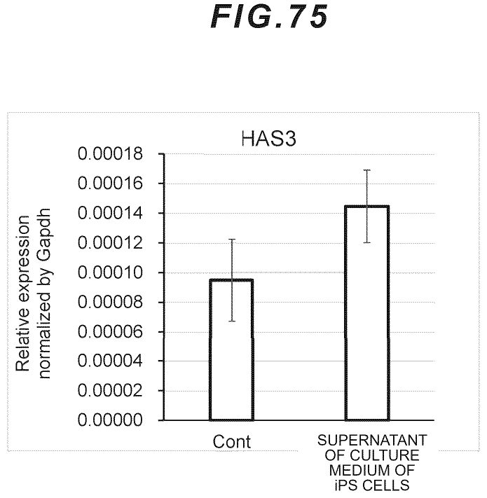

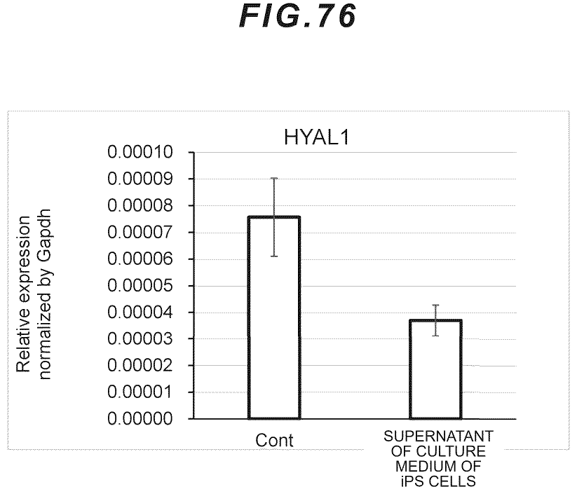

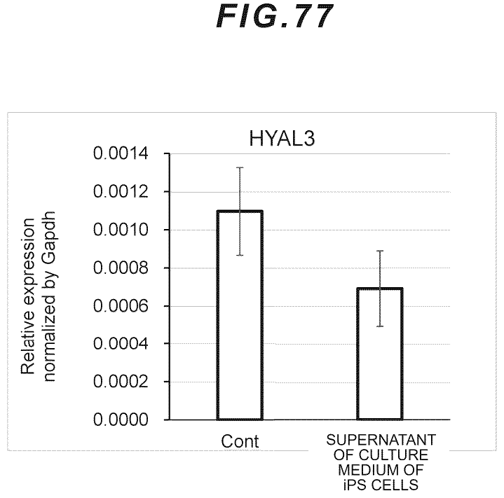

Fibroblasts were cultured and treated with the supernatant from the culture medium as in Example 8. The RNA expression levels of the hyaluronic acid synthase enzymes HAS1, HAS2, and HAS3, along with the hyaluronic acid-degrading enzymes HYAL1 and HYAL3, were analyzed following the same procedure as in Example 8. As shown in FIGS. 73, 74, and 75, fibroblasts treated with the culture supernatant from Example 7 exhibited increased expression levels of the hyaluronic acid synthase enzymes HAS1, HAS2, and HAS3 compared to the control group. Additionally, as shown in FIGS. 76 and 77, the expression levels of the hyaluronic acid-degrading enzymes HYAL1 and HYAL3 decreased in the fibroblasts treated with the culture supernatant from Example 7. These findings indicate that the culture medium supernatant may serve as a promoter of extracellular matrix production, an inhibitor of extracellular matrix degradation, a rejuvenating agent, and an agent for mitigating age-related changes.

1.-22. (canceled)

23. An anti-aging agent comprising a stem cell, an extract of a stem cell, a medium of a stem cell, a supernatant of a culture medium during stem cell induction, a supernatant of a culture medium of stem cells, or a secretion of a stem cell.

24. A rejuvenation agent comprising a stem cell, an extract of a stem cell, a medium of a stem cell, a supernatant of a culture medium during stem cell induction, a supernatant of a culture medium of stem cells, or a secretion of a stem cell.

25. An amplifier for an expression of a rejuvenation factor or a rejuvenation-related factor comprising a stem cell, an extract of a stem cell, a medium of a stem cell, a supernatant of a culture medium during stem cell induction, a supernatant of a culture medium of stem cells, or a secretion of a stem cell.

26.-29. (canceled)
